(19) United States
(12) Patent Application Publication
(10) Pub. No.: US 2010/0016307 A1
(43) Pub. Date: Jan. 21, 2010

(54) NOVEL COMPOUNDS

(76) Inventors: Toshihiro Hamajima, Ibaraki (JP); Hiroko Nakamura, Ibaraki (JP); Jun Tang, Durham, NC (US)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 12/446,762

(22) PCT Filed: Oct. 24, 2007
(86) PCT No.: PCT/EP07/61412

§ 371 Date: Apr. 23, 2009

Related U.S. Application Data
(60) Provisional application No. 60/863,139, filed on Oct. 27, 2006.

(51) Int. Cl. A61K 31/5377; C07D 471/02; C07D 413/14; A61K 31/437

(52) U.S. Cl. 514/234.5; 546/113; 544/127; 514/300

(57) ABSTRACT

The present invention relates to compounds of formula (I):

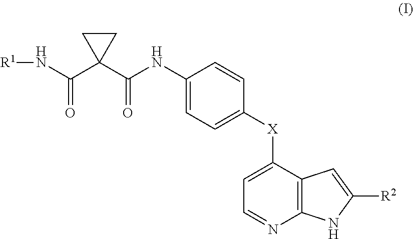

and processes for preparing them, compositions containing them and their use in treating diseases relating to inappropriate c-Met activity.

### FIELD OF THE INVENTION

The present invention relates to novel 7-azaindoles derivatives (or salts or solvates thereof) that are histamine c-Met kinase inhibitors. Such compounds (or salts or solvates thereof) may be useful in the treatment of various disorders in particular cancer, various cardiovascular diseases, rheumatoid arthritis, malaria and other disorders described herein that are associated with inappropriate c-Met or HGF activity.

### BACKGROUND OF THE INVENTION

Protein kinases represent a large family of enzymes that catalyse the phosphorylation of proteins, and play a central role in the regulation of a wide variety of cellular processes. Abnormal protein kinase activity has been related to a plethora of disorders ranging from diseases such as psoriasis to virulent diseases such as glioblastoma (brain cancer).

Kinases and their ligands play critical roles in various cellular activities. Thus deregulation of kinase enzymatic activity can lead to altered cellular properties such as uncontrolled cell growth that is associated with cancers. A number of pathological diseases have been linked to altered kinase signalling, including immunological disorders and degenerative, inflammatory and cardiovascular diseases. Therefore the kinase enzyme family has become an important and interesting therapeutic target.

The hepatocyte growth factor receptor (“HGFR” or “c-Met”) is a receptor tyrosine kinase (RTK) which is an attractive target for oncological, antiangiogenic and antiproliferative activity [Birchmeier et al, Nature Reviews, 4:915-925 (2003)]. c-Met RTK is encoded by the Met proto-oncogene. It is a member of a subfamily of heterodimeric RTKs which include Met, Ron and Sea. c-Met is expressed in numerous tissues such as epithelial, endothelial and mesenchymal cells, although primarily cells of epithelial origin [Maulik et al., Cytokine and Growth Factor Rev., 13:41-59, (2002)].

Activation of the c-Met receptor induces proliferation, motility, invasion and angiogenesis. It has also been shown to be important in morphogenic differentiation and organisation of three-dimensional tubular structures, for example gland formation and renal tubular cells [Ma et al., Cancer and Metastasis Rev., 22:309-325, (2003)].

The endogenous ligand for c-Met is hepatocyte growth factory (HGF), also known as “scatter factor” (SF). HGF is a heterodimeric protein which is secreted by mescenchymal or stromal cells and is a potent inducer of angiogenesis and survival factor for endothelial cells [Bussolino et al., J. Cell Biol., 119(3):629-642, (1992), Birchmeier et al. Trends Cell Biol, 8:404-410 (1998)]. For an in-depth review and discussions on HGF and c-Met interactions see Goldberg and Rosen, “Hepatocyte Growth Factor-Scatter Factor and the c-Met Receptor”, Birkhauser Verlag-Basel, (1993).

Various biological activities have been reported for HGF through its interactions with c-Met. Binding of HGF induces activation of c-Met via autophosphorylation which results in an increase of receptor-dependent signalling which consequently promotes cell growth and invasion. Thus, signal transduction through the activation of the c-Met receptor is responsible for many of the characteristics of tumour cells.

Both HGF and c-Met are expressed at abnormally high levels in a number of human cancers (particularly sarcomas). For tumour growth to occur, new blood vessels must be recruited into the tumour from pre-existing vessels in conjunction with invasion, adhesion and proliferation of malignant cells. c-Met gene amplification, mutation and rearrangement have also been observed in a subset of human cancers. Activating mutations in the kinase domain of the c-Met gene have been implicated as the cause of hereditary papillary renal carcinoma and have been observed in sporatic papillary renal carcinoma, ovarian cancer, childhood hepatocellular carcinoma, gastric cancer, lung cancer and squamous cell carcinoma [Langati et al., Curr. Drug Targets, 2:41-55, (2001), Danilkovitch-Miagkova et al., J. Clin. Invest. 109:863-867 (2002)]. Numerous studies have correlated the expression of c-Met and/or HGF with disease progression in a variety of tumour types including breast, colon, renal, lung, prostate, pancreas, brain, liver, ovaries, bone, stomach, skin bladder and gall bladder cancers in addition to squamous cell myeloid leukaemia, hemangiomas, melanomas, astrocytomas and glioblastomas. Furthermore, over expression of the c-Met oncogene has also been suggested to play a role in the progression and pathogenesis of in a number of human cancers, such as thyroid tumours [Oncogene, 7:2549-2553, (1992)].

Inhibition of angiogenesis has been shown to be linked to the suppression or reversion of tumour progression [Boehm et al., Nature, 390:404-407, (1997)], especially if multiple inhibitors are employed compared to just one. Angiogenesis can be stimulated by HGF as well as vascular endothelial growth factor (VEGF) and basic fibroblast growth factor (bFGF). Thus, modulation of c-Met is desirable as a means to treat cancer and cancer-related diseases.

Angiogenesis is the development of new blood vessels, generally capillaries from pre-existing vasculature. Arteriogenesis is the process of remodelling small vessels into larger conduit vessels. These processes of vascular growth are required during beneficial processes such as tissue repair, wound healing and at certain stages of the female reproductive cycle. Inappropriate angiogenesis has been associated with several disease states including retionopathies, ischemic disease, neoplasias, rheumatoid arthritis, psoriasis, artherosolerosis, certain forms of chronic inflammatory disorders and certain forms of mascular degeneration [Middleton et al., Arthritis Res. Ther., 6(2):60-72, (2004)]. The inhibition of angiogenesis may result in blocking the development of pathological pannus tissue in rheumatoid arthritis.

Stimulation of vascular growth has potential utility for treatment of ischemia-induced pathologies such as myocardial infarction, coronary artery disease, stroke and peripheral vascular disease [Ono et al., Circulation, 95:2552-2558, (1997)]. The sprouting of new vessels and/or the expansion of smaller vessels in ischemic tissues prevents the death of ischemic tissue and encourages tissue repair. Certain diseases are well-known to be associated with deregulated angiogenesis such as retinopathies (including diabetic retinopathy) ocular neovascularisation, psoriasis, hemangioma, hermangioblastoma, age-related macular degeneration, arteriosclerosis, inflammatory disease for example rheumatoid or rheumatic inflammatory disease especially arthritis (including rheumatoid arthritis) or other chronic inflammatory disorders such as chronic asthma, arterial or post-transplantational atherosclerosis, endometriosis and neoplastic diseases such as so-called solid tumours and liquid tumours (e.g. leukemias). For a discussion on the role of angiogenesis in a various disease states see, for example. Fan et. al. Trends in Pharm. Sci., 16:54-66; Shawver et. al., DDT Vol. 2(2), (1997); Folkman, Nature Medicine, 1:27:31, (1995). Felmeden et al., European Heart Journal, 24:586-603 (2002).

Other non-oncological diseases and disorders that have been linked to elevated levels of c-Met and HGF include hypertension rheumatoid arthritis and myocardial infarction. Increased levels of HGF have been observed in patients with hepatic failure [Gohda et al., Exp. Cell Res., 166:139-150 (1986)] and it has been shown to be a mitogen for certain cell types such as melanocytes, keratinocytes, renal tubular cells, cells of epithelial origin and certain endothelial cells [Igawa et al., Biochem. Biophys. Res. Comm., 174(2):831-838 (1991)]. The c-Met oncogene has postulated to play a role in microglial reactions to CNS injuries [Oncogene, 8:219-222, (1993)].

Plasmodium, the causative agent of malaria causes an increase in HGF secretion. Inhibition of the c-Met kinase has also been shown to induce a specific increase in apoptosis of infected cells and thus a significant decrease in infection [Leirinao et. al., Cell. Microbiol., 7(4):603-609, (2005)] Infection with Helicobacter pylori is assumed to lead to invasive gastric cancer, and has also been shown to activate c-Met [Churin et al., J. Cell Bio., 161(2):249-255, (2003)].

Therefore c-Met inhibitors may be useful in treating diseases such as cancer and other diseases related to abnormal cell growth and c-Met activation.

### BRIEF SUMMARY OF THE INVENTION

In one aspect of the present invention, there is provided a compound of Formula (I):

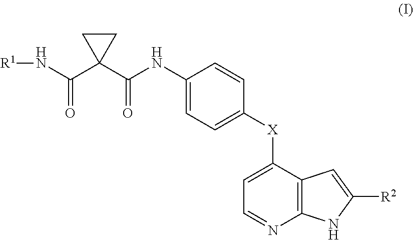

wherein:

R¹ represents aryl, in which said aryl may be optionally substituted with one two or three substituents independently selected from C₁₋₆alkyl, C₁₋₆alkoxy, halogen, hydroxy or C₁₋₃haloalkyl;

R² represents hydrogen, aryl, —COOR³ or C(O)NR⁴R⁵, in which said aryl may be optionally substituted with one two or three substituents independently selected from C₁₋₆alkyl, C₁₋₆alkoxy, halogen, hydroxy or C₁₋₃haloalkyl;

X represents NH or O;

R³ represents hydrogen or C₁₋₆alkyl;

R⁴ represents hydrogen or C₁₋₆alkyl;

R⁵ represents —C₁₋₃alkylNR⁶R⁷, —C₁₋₃alkyl-SO₂—C₁₋₃alkyl, —C₁₋₃alkylOH, —C₁₋₃alkyl-C(O)NH₂, —C₁₋₃alkylheteroaryl, —C₀₋₃alkylaryl (in which said aryl may be optionally substituted with one two or three substituents independently selected from C₁₋₆alkyl, C₁₋₆alkoxy, halogen, hydroxy, C₁₋₃haloalkyl or —C₀₋₃alkylNR⁸R⁹), or C₃₋₆cycloalkyl;

R⁶ and R⁷ are each independently selected from hydrogen, C₁₋₃alkyl or together may form a 4-7 membered saturated heterocyclic ring, optionally in which one or two carbon atoms may be replaced with either oxygen or nitrogen;

R⁸ and R⁹ are each independently selected from hydrogen or C₁₋₆alkyl;

or a salt or solvate thereof.

In a second aspect of the present invention, there is provided a compound of formula (I), or a pharmaceutically acceptable salt or solvate thereof for use in therapy, and particularly in the treatment of disorders mediated by inappropriate c-Met activity, such as cancer, certain viral diseases and cardiovascular disorders.

In a third aspect of the present invention, there is provided a pharmaceutical composition comprising a compound of formula (I) or a pharmaceutically acceptable salt or solvate thereof, optionally with one or more pharmaceutically acceptable carriers, diluents and excipients.

In a fourth aspect of the present invention, there is provided a combination comprising a compound of formula (I) or a pharmaceutically acceptable salt or solvate thereof and one or more therapeutic agents, such as one or more anti-cancer agents, e.g. one or more antineoplastic agents.

In a fifth aspect of the present invention, there is provided the use of a compound of formula (I), or a pharmaceutically acceptable salt or solvate thereof in the preparation of a medicament for use in the treatment of a disorder mediated by inappropriate c-Met activity, such as cancer, certain viral diseases and cardiovascular disorders.

In a sixth aspect of the present invention, there is provided the use of a compound of formula (I), or a pharmaceutically acceptable salt or solvate thereof in combination with one or more therapeutic agents (such as one or more anti-cancer agents, e.g. one or more antineoplastic agents) in the preparation of a medicament for use in the treatment of a disorder mediated by inappropriate c-Met activity, such as cancer, certain viral diseases and cardiovascular disorders.

In a seventh aspect of the present invention, there is provided a method of treating a disorder in a mammal, said disorder being mediated by inappropriate c-Met activity, such as cancer, certain viral diseases and cardiovascular disorders including: administering to said mammal a compound of formula (I) or a pharmaceutically acceptable salt or solvate thereof.

In a eighth aspect of the present invention, there is provided a method of treating cancer in a mammal, including administering to said mammal a compound of formula (I), or a pharmaceutically acceptable salt or solvate thereof.

In an ninth aspect of the present invention, there is provided a method of treating cancer in a mammal, including administering to said mammal (i) a compound of formula (I), or a pharmaceutically acceptable salt or thereof and (ii) at least one additional anti-cancer therapy.

In a tenth aspect of the present invention, there is provided processes for the synthesis of compounds of formula (I) and salts or solvates thereof.

### DETAILED DESCRIPTION OF THE INVENTION

The inappropriate c-Met activity referred to herein is any c-Met activity that deviates from the normal c-Met activity expected in a particular mammalian subject. Inappropriate c-Met activity may take the form of, for instance, an abnormal increase in activity, or an aberration in the timing and or control of c-Met activity. Such inappropriate activity may result then, for example, from overexpression or mutation of the protein kinase or ligand leading to inappropriate or uncontrolled activation of the receptor. Furthermore, it is also understood that unwanted c-Met activity may reside in an abnormal source, such as a malignancy. That is, the level of c-Met activity does not have to be abnormal to be considered inappropriate, rather the activity derives from an abnormal source. In a like manner, the inappropriate angiogenesis referred to herein is any angiogenic activity that deviates from the normal angiogenic activity expected in a particular mammalian subject. Inappropriate angiogenesis may take the form of, for instance, an abnormal increase in activity, or an aberration in the timing and or control of angiogenic activity. Such inappropriate activity may result then, for example, from overexpression or mutation of a protein kinase or ligand leading to inappropriate or uncontrolled activation of angiogenesis. Furthermore, it is also understood that unwanted angiogenic activity may reside in an abnormal source, such as a malignancy. That is, the level of angiogenic activity does not have to be abnormal to be considered inappropriate, rather the activity derives from an abnormal source.

C₁₋₆alkyl, whether alone or as part of another group, may be straight chain or branched. Representative examples include methyl, ethyl, n-propyl, iso-propyl, n-butyl, sec-butyl, iso-butyl, t-butyl, n-pentyl, neo-pentyl and n-hexyl. Exemplary alkyl groups are C₁₋₃alkyl, such as methyl, ethyl, n-propyl and iso-propyl, but particularly methyl.

C₁₋₆alkoxy is used to describe —O—C₁₋₆alkyl, where the alkyl is as defined herein above. Representative C₁₋₆alkoxy groups include methoxy, ethoxy, n-propoxy, iso-propoxy, n-butoxy, sec-butoxy, iso-butoxy, t-butoxy, n-pentyloxy, neo-pentyloxy and n-hexyloxy. Exemplary alkoxy groups are C₁₋₃alkoxy such as methoxy, ethoxy, n-propoxy, iso-propoxy, but particularly methoxy.

C₃₋₆cycloalkyl refers to a non-aromatic cyclic hydrocarbon ring having from three to six carbon atoms. Representative examples include cyclopropyl, cyclobutyl, cyclopentyl and cyclohexyl.

The term “halogen” is used herein to describe, unless otherwise stated, a group selected from fluorine, chlorine, bromine or iodine. Particular halogen atoms are chlorine and fluorine.

As used herein, the term “hydroxy” refers to the group —OH.

As used herein, the term “C₁-C₃haloalkyl” refers to a straight or branched chain hydrocarbon containing at least 1, and at most 3, carbon atoms substituted with at least one halogen, halogen being as defined herein. Examples of branched or straight chained “C₁-C₃haloalkyl” groups useful in the present invention include, but are not limited to, methyl, ethyl, propyl and isopropyl, substituted independently with one or more halogen atoms, e.g. fluoro, chloro, bromo and iodo. An exemplary C₁₋₃haloalkyl group is trifluoromethyl.

The term “aryl” includes single e.g. phenyl and fused aromatic rings such as anthracene, phenanthrene, chrysene, perylene, pyrene, naophthylene. Representative aryl groups include phenyl and naphthyl.

The term “heterocyclic” or “heterocycle” is intended to mean a 4-7 membered monocyclic saturated ring containing between 1 and 3 heteroatoms selected from O, N, SO₂ and S, or a 4-7 membered saturated ring, containing between 1 and 3 heteroatoms selected from O, N, SO₂ and S fused to a benzene ring. Suitable examples of such monocyclic rings include azetidinyl, pyrrolidinyl, piperidinyl, piperazinyl, morpholinyl, thiomorpholinyl, tetrahydrofuranyl, pyranyl, 1,4-dioxanyl, 1,3-dioxanyl, tetrahydrothiopyranyl, diazepanyl, azepanyl and azocanyl. Suitable examples of benzofused heterocylic rings include indolinyl, isoindolinyl, benzodioxolyl, dihydroquinolinyl, dihydroisoquinolinyl, dihydrobenzothiopyranyl and dihydrobenzothiopyranyl-1-dioxide.

The term “heteroaryl” is intended to mean a 5-7 membered monocyclic aromatic or a 8-11 membered bicyclic aromatic ring containing 1 to 3 heteroatoms selected from oxygen, nitrogen and sulphur. Suitable examples of such monocyclic aromatic rings include thienyl, furyl, pyrrolyl, triazolyl, tetrazolyl, imidazolyl, oxazolyl, thiazolyl, oxadiazolyl, and isothiazolyl, isoxazolyl, thiadiazolyl, pyrazolyl, pyrimidyl, pyridazinyl, pyrazinyl, pyridyl, and triazolyl. Suitable examples of such 8-11 membered bicyclic aromatic rings include furopyridinyl and pyrazolopyrimidyl, and benzofused aromatic rings such as quinolinyl, isoquinolinyl, quinazolinyl, quinoxalinyl, cinnolinyl, naphthyridinyl, indolyl, indazolyl, pyrrolopyridinyl, benzofuranyl, benzothienyl, benzimidazolyl, benzoxazolyl, benzisoxazolyl, benzothiazolyl, benzisothiazolyl, benzotriazolyl, benzoxadiazolyl, benzothiadiazolyl and the like. Particular heteroaryl groups include pyridyl, isoxazolyl, pyrazolyl, oxazolyl, triazolyl, pyrazolopyrimidyl, indolyl, quinolinyl, benzothiazolyl and benzotriazolyl.

As used herein, the term “optionally” means that the subsequently described event(s) may or may not occur, and includes both event(s), which occur, and events that do not occur.

It is to be understood that the present invention covers the compounds of formula (I) as the free base and as salts and solvates thereof, for example a pharmaceutically acceptable salt or solvate.

It is to be further understood that references hereinafter to compounds of the invention or to compounds of formula (I) means a compound of formula (I) as the free base, or as a salt, or as a solvate.

The compound of the present invention may be in the form of and/or may be administered as a pharmaceutically acceptable salt. Pharmaceutically acceptable salts include acid and base addition salts. For a review on suitable salts see Berge et al., J. Pharm. Sci., 66:1-19, (1977).

Typically, a pharmaceutically acceptable salt refers to non-toxic salts of the compounds of this invention which may be readily prepared by using a desired acid as appropriate. The salt may precipitate from solution and be collected by crystallization and filtration or may be recovered by evaporation of the solvent.

A pharmaceutically acceptable acid addition salt can be formed by reaction of a compound of formula (I) with a suitable inorganic or organic acid (such as hydrobromic, hydrochloric, formic, sulfuric, nitric, phosphoric, succinic, maleic, acetic, fumaric, citric, tartaric, benzoic, p-toluenesulfonic, methanesulfonic or naphthalenesulfonic acid), optionally in a suitable solvent such as an organic solvent, to give the salt. Thus, a pharmaceutically acceptable acid addition salt of a compound of formula (I) can be for example a hydrobromide, hydrochloride, formate, sulfate, nitrate, phosphate, succinate, maleate, acetate, fumarate, citrate, tartrate, benzoate, p-toluenesulfonate, methanesulfonate or naphthalenesulfonate salt. Other representative salts include the following salts: benzenesulfonate, bicarbonate, bisulfate, bitartrate, borate, bromide, calcium edetate, camsylate, carbonate, chloride, clavulanate, dihydrochloride, edetate, edisylate, estolate, esylate, gluceptate, gluconate, glutamate, glycollylarsanilate, hexylresorcinate, hydrabamine, hydroxynaphthoate, iodide, isethionate, lactate, lactobionate, laurate, malate, mandelate, mesylate, methylbromide, methylnitrate, methylsulfate, monopotassium maleate, mucate, napsylate, N-methylglucamine, oxalate, pamoate (embonate), palmitate, pantothenate, diphosphate, polygalacturonate, potassium, salicylate, sodium, stearate, subacetate, tannate, teoclate, tosylate, triethiodide, trimethylammonium and valerate.

Suitable pharmaceutically acceptable base salts include ammonium salts, alkali metal salts such as those of sodium and potassium, alkaline earth metal salts such as those of calcium and magnesium, and salts with organic bases, including salts of primary, secondary and tertiary amines such as isopropylamine, diethylamine, ethanolamine, trimethylamine, dicyclohexylamine and N-methyl-D-glucamine.

Other non-pharmaceutically acceptable salts, e.g. oxalates or trifluoroacetates, may be used, for example in the isolation of the compound of the invention, and are included within the scope of this invention. The invention includes within its scope all possible stoichiometric and non-stoichiometric forms of the salts of the compounds of formula (I).

It will be appreciated that many organic compounds can form complexes with solvents in which they are reacted or from which they are precipitated or crystallized. These complexes are known as “solvates”. For example, a complex with water is known as a “hydrate”. Solvents with high boiling points and/or solvents with a high propensity to form hydrogen bonds such as water, xylene, N-methylpyrrolidinone methanol may be used to form solvates. Methods for identification of solvates include, but are not limited to, NMR and microanalysis. Solvates of the compound of the invention are within the scope of the invention.

The compounds of formula (I) may be in crystalline or amorphous form. Furthermore, some of the crystalline forms of the compounds of formula (I) may exist as polymorphs, which are included within the scope of the present invention. The most thermodynamically stable polymorphic form of compounds of formula (I) is of particular interest.

Polymorphic forms of compounds of formula (I) may be characterized and differentiated using a number of conventional analytical techniques, including, but not limited to, X-ray powder diffraction (XRPD) patterns, infrared (IR) spectra, Raman spectra, differential scanning calorimetry (DSC), thermogravimetric analysis (TGA) and solid state nuclear magnetic resonance (NMR).

It will be appreciated that compounds of formula (I) may possess one or more asymmetric carbon atoms so that optical isomers e.g. enantiomers or diastereoisomers may be formed. The present invention encompasses all optical and geometric isomers of the compounds of formula (I) whether as individual isomers isolated such as to be substantially free of the other isomer (i.e. pure) or as mixtures thereof (i.e. racemates and racemic mixtures). Geometric isomers may occur, for example, in rigidified ring systems or around double bonds, where cis and trans isomerism may occur. An individual isomer isolated such as to be substantially free of the other isomer (i.e. pure) may be isolated such that less than about 10%, particularly less than about 1%, for example less than about 0.1% of the other isomer is present.

Further, it will be appreciated that the R and S enantiomers may be isolated from the racemate by conventional resolution methods such as preparative HPLC involving a chiral stationary phase, by resolution using fractional crystallisation of a salt of the free base with a chiral acid, by chemical conversion to a diastereoisomer using a chiral auxiliary followed by chromatographic separation of the isomers and then removal of the chiral auxiliary and regeneration of the pure enantiomer, or by total asymmetric synthesis.

Certain compounds of formula (I) may exist in one of several tautomeric forms. It will be understood that the present invention encompasses all tautomers of the compounds of formula (I) whether as individual tautomers or as mixtures thereof.

Certain compounds of formula (I) may exist in a zwitterionic form. Zwitterions are compounds which contain both acidic and basic groups in different parts of the same molecule. Zwitterions carry a neutral charge overall. At neutral pH most zwitterions are therefore negatively charged anions (such as a carboxy group) and positively charged cations (such as amines) at the same time.

It will be appreciated from the foregoing that included within the scope of the invention are all solvates, hydrates, complexes, isomers and polymorphic forms of the compound of the invention and salts thereof.

In another aspect of the present invention X represents 0. In another aspect, X represents NH.

In a further aspect, the present invention provides a compound of formula (I) in which R¹ represents phenyl, in which said phenyl may be optionally substituted with one or two e.g. one substituent(s) independently selected from C₁₋₃alkyl e.g. methyl, C₁₋₃alkoxy e.g. methoxy, halogen such as chlorine or fluorine e.g. fluorine, hydroxy or C₁₋₃haloalkyl e.g. trifluoromethyl.

In another aspect of the invention, R¹ represents phenyl, in which said phenyl may be optionally substituted with one substituent selected from methyl, methoxy, chlorine, fluorine, hydroxy or trifluoromethyl and R² is either —COOR³ or C(O)NR⁴R⁵, wherein R³, R⁴ and R⁵ are as previously described.

In another aspect of the invention, R¹ represents phenyl, in which said phenyl may be optionally substituted with one substituent selected from methyl, methoxy, chlorine, fluorine, hydroxy or trifluoromethyl and R² is C(O)NR⁴R⁵, wherein R⁴ and R⁵ are as previously described.

In another aspect of the present invention, R¹ represents phenyl substituted in the 4-position (para) with fluorine.

In another aspect of the present invention, R² represents unsubstituted aryl, such as phenyl, —COOR³ or C(O)NR⁴R⁵, wherein R³, R⁴ and R⁵ are as previously described.

In another aspect, R² represents C(O)NR⁴R⁵, wherein R⁴ and R⁵ are as previously described.

In a further aspect, R³ represents hydrogen or C₁₋₆alkyl such as methyl or ethyl e.g. ethyl.

In yet another aspect of the present invention, R⁴ represents hydrogen or C₁₋₃alkyl such as methyl or ethyl e.g. methyl. In another aspect, R⁴ represents hydrogen.

In a further aspect of the invention, R⁴ represents hydrogen.

In another aspect of the present invention, there is provided a compound of formula (I) in which R⁵ represents —C₁₋₃alkylNR⁶R⁷, —C₁₋₃alkyl-SO₂—C₁₋₃alkyl, —C₁₋₃alkylOH, —C₁₋₃alkyl-C(O)NH₂, —C₁₋₃alkylheteroaryl e.g. —C₁₋₃alkylimidazole, —C₀₋₃alkylphenyl (in which said phenyl may be optionally substituted with one or two, e.g. one substituents(s) selected from C₁₋₃alkyl e.g. methyl, C₁₋₃alkoxy e.g. methoxy, halogen, hydroxy, C₁₋₃haloalkyl e.g. trifluoromethyl or —C₀₋₃alkylNR⁸R⁹), cyclopropane or cyclobutane, and R⁶, R⁷, R⁸ and R⁹ are as previously described.

In another aspect, the aryl or phenyl groups of R⁵ may be substituted with one or two, e.g. one substituents(s) independently selected from methyl, methoxy, fluorine, chlorine, hydroxy, trifluoromethyl or —C₀₋₃alkylNR⁸R⁹, and R⁸ and R⁹ are as previously described.

Representative examples of R⁵ include: 2-(dimethylamino)ethyl, 2-(methylsulfonyl)ethyl, 2-(4-morpolinyl)ethyl, 2-hydroxyethyl, 2-amino-2-oxoethyl, 2-(1H-imadozl-4-yl)ethyl, 2-[(3-hydroxy-4-(methyloxy)phenyl]ethyl, [4-(dimethylamino)phenyl]methyl, [2-(methyloxy)phenyl]methyl, 2-(methyloxy)phenyl, 3-[(dimethylamino)methyl]phenyl, 3-(dimethylamino)propyl, 2-(1-piperazinyl)ethyl or cyclopropyl groups.

In yet another aspect of the present invention R⁶ and R⁷ are each independently selected from hydrogen, C₁₋₃alkyl or together may form a 5-7 membered ring, optionally in which one or two carbon atoms may be replaced with either oxygen or nitrogen.

In another aspect, R⁶ and R⁷ are each independently selected from hydrogen, methyl or together may form a 6-membered ring, optionally in which one carbon atom may be replaced with either oxygen or nitrogen, to form for example morpholine or piperazine, e.g. morpholine.

In another aspect, R⁸ and R⁹ are each independently selected from hydrogen, methyl or ethyl.

In another aspect, R⁸ and R⁹ are each independently selected from hydrogen or methyl.

Representative compounds according to the invention may include Examples E1 to E24, and salts or solvates thereof, particularly pharmaceutically acceptable salts or solvates thereof.

A particular compound of the invention is N¹-(4-fluorophenyl)-N¹-(4-{[2-({[2-(4-morpholinyl)ethyl]amino}carbonyl)-1H-pyrrolo[2,3-b]pyridin-4-yl]oxy}phenyl)-1,1-cyclopropanedicarboxamide and salts or solvates thereof, particularly pharmaceutically acceptable salts or solvates thereof.

It is to be understood that the invention includes all possible combinations of groups and substituents described herein.

Examples of disease states in which compounds of formula (I), or pharmaceutically acceptable salts or solvates thereof may have potentially beneficial antitumour effects include, but are not limited to, cancers of the lung, bone, pancreas, skin, head, neck, uterus, ovaries, stomach, colon, breast, esophagus, small intestine, bowel, endocrine system, thyroid glad, parathyroid gland, adrenal gland, urethra, prostate, penis, testes, ureter, bladder, kidney or liver; rectal cancer; cancer of the anal region; carcinomas of the fallopian tubes, endometrium, cervix, vagina, vulva, renal pelvis, renal cell; sarcoma of soft tissue; myxoma; rhabdomyoma; fibroma; lipoma; teratoma; cholangiocarcinoma; hepatoblastoma; angiosarcoma; hemagioma; hepatoma; fibrosarcoma; chondrosarcoma; myeloma; chronic or acute leukemia; lymphocytic lymphomas; primary CNS lymphoma; neoplasms of the CNS; spinal axis tumours; squamous cell carcinomas; synovial sarcoma; malignant pleural mesotheliomas; brain stem glioma; pituitary adenoma; bronchial adenoma; chondromatous hanlartoma; inesothelioma; Hodgkin's Disease or a combination of one or more of the foregoing cancers.

The compounds of the present invention may also be useful in the treatment of one or more diseases afflicting mammals which are characterized by cellular proliferation in the area of disorders associated with neo-vascularization and/or vascular permeability including blood vessel proliferative disorders including arthritis (rheumatoid arthritis) and restenosis; fibrotic disorders including hepatic cirrhosis and atherosclerosis; mesangial cell proliferative disorders include glomerulonephritis, diabetic nephropathy, malignant nephrosclerosis, thrombotic microangiopathy syndromes, proliferative retinopathies, organ transplant rejection and glomerulopathies; and metabolic disorders include psoriasis, diabetes mellitus, chronic wound healing, inflammation and neurodegenerative diseases.

Furthermore, the compounds of the invention may be of use in the treatment of viral diseases related to activation of c-Met kinase, including, but not limited to malaria and Helicobacter pylori infection.

Further conditions include cardiovascular disorders, such as myocardial infarction, coronary artery disease, stroke and peripheral vascular disease.

Therefore, there is provided a compound of formula (I), or a pharmaceutically acceptable salt or solvate thereof for use in therapy, and particularly in the treatment of disorders mediated by inappropriate c-Met activity, such as cancer, certain viral diseases and cardiovascular disorders.

While it is possible that, for use in therapy, therapeutically effective amounts of a compound of formula (I), as well as pharmaceutically acceptable salts or solvates thereof, may be administered as the raw chemical, it is possible to present the active ingredient as a pharmaceutical composition. The compounds of the formula (I) and pharmaceutically acceptable salts or solvates thereof are as described above. The carrier(s), diluent(s) or excipient(s) must be acceptable in the sense of being compatible with the other ingredients of the composition and not deleterious to the recipient thereof. In accordance with another aspect of the invention there is also provided a process for the preparation of a pharmaceutical composition including admixing a compound of the formula (I), or pharmaceutically acceptable salts or solvates thereof, with one or more pharmaceutically acceptable carriers, diluents or excipients.

Therefore, there is provided a pharmaceutical composition comprising a compound of formula (I) or a pharmaceutically acceptable salt thereof and one or more pharmaceutically acceptable carriers, diluents and excipients.

Pharmaceutical compositions may be presented in unit dose forms containing a predetermined amount of active ingredient per unit dose. Such a unit may contain, for example, 0.5 mg to 1 g, preferably 1 mg to 700 mg, of a compound of the formula (I) depending on the condition being treated, the route of administration and the age, weight and condition of the patient. Preferred unit dosage compositions are those containing a daily dose or sub-dose, as herein above recited, or an appropriate fraction thereof, of an active ingredient. Furthermore, such pharmaceutical compositions may be prepared by any of the methods well known in the pharmacy art.

Pharmaceutical compositions may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such compositions may be prepared by any method known in the art of pharmacy, for example by bringing into association the active ingredient with the carrier(s) or excipient(s).

Pharmaceutical compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the active drug component can be combined with an oral, non-toxic pharmaceutically acceptable inert carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

Capsules are made by preparing a powder mixture as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of the compound. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additives such as peppermint oil or natural sweeteners or saccharin or other artificial sweeteners, and the like can also be added.

Where appropriate, dosage unit compositions for oral administration can be microencapsulated. The composition can also be prepared to prolong or sustain the release as for example by coating or embedding particulate material in polymers, wax or the like.

The compounds of formula (I) and pharmaceutically acceptable salts or solvates thereof can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The compounds of formula (I) and pharmaceutically acceptable salts or solvates thereof may also be delivered by the use of monoclonal antibodies as individual carriers to which the compound molecules are coupled. The compounds may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxidepolylysine substituted with palmitoyl residues. Furthermore, the compounds may be coupled to a class of biodegradable polymers useful in achieving controlled release of a drug, for example, polylactic acid, polepsilon caprolactone, polyhydroxy butyric acid, polyorthoesters, polyacetals, polydihydropyrans, polycyanoacrylates and cross-linked or amphipathic block copolymers of hydrogels.

Pharmaceutical compositions adapted for transdermal administration may be presented as discrete patches intended to remain in intimate contact with the epidermis of the recipient for a prolonged period of time. For example, the active ingredient may be delivered from the patch by iontophoresis as generally described in Pharm. Res., 3(6):318 (1986).

Pharmaceutical compositions adapted for topical administration may be formulated as ointments, creams, suspensions, lotions, powders, solutions, pastes, gels, sprays, aerosols or oils.

For treatments of the eye or other external tissues, for example mouth and skin, the compositions are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical compositions adapted for topical administrations to the eye include eye drops wherein the active ingredient is dissolved or suspended in a suitable carrier, especially an aqueous solvent.

Pharmaceutical compositions adapted for topical administration in the mouth include lozenges, pastilles and mouth washes.

Pharmaceutical compositions adapted for rectal administration may be presented as suppositories or as enemas.

Pharmaceutical compositions adapted for nasal administration wherein the carrier is a solid include a coarse powder having a particle size for example in the range 20 to 500 microns which is administered in the manner in which snuff is taken, i.e., by rapid inhalation through the nasal passage from a container of the powder held close up to the nose. Suitable compositions wherein the carrier is a liquid, for administration as a nasal spray or as nasal drops, include aqueous or oil solutions of the active ingredient.

Pharmaceutical compositions adapted for administration by inhalation include fine particle dusts or mists, which may be generated by means of various types of metered, dose pressurised aerosols, nebulizers or insufflators.

Pharmaceutical compositions adapted for vaginal administration may be presented as pessaries, tampons, creams, gels, pastes, foams or spray compositions.

Pharmaceutical compositions adapted for parenteral administration include aqueous and non-aqueous sterile injection solutions which may contain anti-oxidants, buffers, bacteriostats and solutes which render the composition isotonic with the blood of the intended recipient; and aqueous and non-aqueous sterile suspensions which may include suspending agents and thickening agents. The compositions may be presented in unit-dose or multi-dose containers, for example sealed ampules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

It should be understood that in addition to the ingredients particularly mentioned above, the compositions may include other agents conventional in the art having regard to the type of composition in question, for example those suitable for oral administration may include flavouring agents.

A therapeutically effective amount of a compound of the present invention will depend upon a number of factors including, for example, the age and weight of the animal, the precise condition requiring treatment and its severity, the nature of the composition, and the route of administration, and will ultimately be at the discretion of the attendant physician or veterinarian. However, an effective amount of a compound of formula (I) for the treatment of disorders or diseases associated with inappropriate c-Met activity, will generally be in the range of 0.1 to 100 mg/kg body weight of recipient (mammal) per day and more usually in the range of 1 to 10 mg/kg body weight per day. Thus, for a 70 kg adult mammal, the actual amount per day would usually be from 70 to 700 mg and this amount may be given in a single dose per day or more usually in a number (such as two, three, four, five or six) of sub-doses per day such that the total daily dose is the same. An effective amount of a pharmaceutically acceptable salt thereof may be determined as a proportion of the effective amount of the compound of formula (I) per se. It is envisaged that similar dosages would be appropriate for treatment of the other conditions referred to above.

The compounds of the present invention and their pharmaceutically acceptable salts or solvates may be employed alone or in combination with other therapeutic agents. Particularly, combination with at last one other anti-cancer therapy is envisaged.

Therefore, there is provided a combination comprising a compound of formula (I) or a pharmaceutically acceptable salt thereof and one or more therapeutic agents, such as one or more anti-cancer agents, e.g. one or more antineoplastic agents.

In particular, in anti-cancer therapy, combination with other chemotherapeutic, hormonal or antibody agents is envisaged, as well as combination with surgical therapy and radiotherapy. Combination therapies according to the present invention thus comprise the administration of at least one compound of formula (I) or a pharmaceutically acceptable salt or solvate thereof and the use of at least one other cancer treatment method. For example, combination therapies according to the present invention comprise the administration of at least one compound of formula (I) or a pharmaceutically acceptable salt or solvate thereof and at least one other pharmaceutically active agent, such as an anti-neoplastic agent. The compound(s) of formula (I) and the other pharmaceutically active agent(s) may be administered together or separately and, when administered separately this may occur simultaneously or sequentially in any order. The amounts of the compound(s) of formula (I) and the other pharmaceutically active agent(s) and the relative timings of administration will be selected in order to achieve the desired combined therapeutic effect.

The combinations referred to above may conveniently be presented for use in the form of a pharmaceutical composition and thus pharmaceutical compositions comprising a combination as defined above together with a pharmaceutically acceptable diluent or carrier represent a further aspect of the invention.

The individual compounds of such combinations may be administered either sequentially or simultaneously in separate or combined pharmaceutical compositions. In one embodiment, the individual compounds may be administered simultaneously in a combined pharmaceutical composition. Appropriate doses of known therapeutic agents will be readily appreciated by those skilled in the art.

The compounds of formula (I) or pharmaceutically acceptable salts or solvates thereof and at least one additional cancer treatment therapy may be employed in combination concomitantly or sequentially in any appropriate combination with such other anti-cancer therapies. In one embodiment, the other anti-cancer therapy is at least one additional chemotherapeutic therapy including administration of at least one anti-neoplastic agent. The administration in combination of a compound of formula (I) or pharmaceutically acceptable salts or solvates thereof with other anti-neoplastic agents may be in combination in accordance with the invention by administration concomitantly in (1) a unitary pharmaceutical composition including both compounds or (2) separate pharmaceutical compositions each including one of the compounds. Alternatively, the combination may be administered separately in a sequential manner wherein one anti-neoplastic agent is administered first and the other second or vice versa. Such sequential administration may be close in time or remote in time.

In particular, where the disorder is cancer, combination with at least one other anti-cancer therapy is envisaged. In particular, in anti-cancer therapy, combination with other chemotherapeutic, hormonal or antibody agents is envisaged as well as combination with surgical therapy and radiotherapy. Combination therapies according to the present invention thus comprise the administration of at least one compound of formula (I) or a pharmaceutically acceptable salt or solvate thereof, or a physiologically functional derivative thereof, and the use of at least one other cancer treatment method. Preferably, combination therapies according to the present invention comprise the administration of at least one compound of formula (I) or a pharmaceutically acceptable salt or solvate thereof, or a physiologically functional derivative thereof, and at least one other pharmaceutically active agent, preferably an anti-neoplastic agent. The compound(s) of formula (I)) and the other pharmaceutically active agent(s) may be administered together or separately and, when administered separately this may occur simultaneously or sequentially in any order and by any convenient route. The amounts of the compound(s) of formula (I) and the other pharmaceutically active agent(s) and the relative timings of administration will be selected in order to achieve the desired combined therapeutic effect.

In one embodiment, an other anti-cancer therapy is at least one additional chemotherapeutic therapy. Such chemotherapeutic therapy may include one or more of the following categories of anti-cancer agents.

(i) antiproliferative/antineoplastic drugs and combinations thereof, as used in medical oncology, such as alkylating agents (for example cis-platin, carboplatin, cyclophosphamide, nitrogen mustard, melphalan, chlorambucil, busulphan and nitrosoureas); antimetabolites (for example antifolates such as fluoropyrimidines like 5-fluorouracil and tegafur, raltitrexed, methotrexate, cytosine arabinoside and hydroxyurea; antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristrine, vinblastine, vindesine and vinorelbine and taxoids like taxol and taxotere); and topoisomerase inhibitors (for example epipodophyllotoxins like etoposide and teniposide, amsacrine, topotecan and camptochecin);

(ii) cytostatic agents such as antioestrogens (for example tamoxifen, toremifine, raloxifine, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate) aromatase inhibitors (for example as anastrozole, letrozole, vorazole and exemestane) and inhibitors of 5α-reductase such as finasteride;

(iii) agents which inhibit cancer cell invasion (for example metalloproteinase inhibitors and inhibitors of urokinase plasminogen activator receptor function);

(iv) inhibitors of growth factor function, for example such inhibitors include growth factor antibodies, growth factor receptor antibodies (for example the anti-erbb2 antibody trastuzumab [Herceptin™] and the anti-erbb1 antibody cetuximab [C225], farnesyl transferase inhibitors, tyrosine kinase inhibitors and serine-threonine kinase inhibitors, for example inhibitors of the epidermal growth factor family (for example EGFR family tyrosine kinase inhibitors such as N-(3-chloro-4-fluorophenyl-7-methoxy-6-(3-morpholinoproproxy)quinazolin-4-amine (gefitinib, AZD1839), N-3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinoproproxy)quinazolin-4-amine (CI-1033)), for example inhibitors of the platelet-derived growth factor family and for example inhibitors of the hepatocyte growth factor family;

(v) antiangiogenic agents such as those which inhibit the effects of vascular edothelial growth factor, (for example the anti-vascular endothelial cell growth factor antibody bevacizumab [Avastin™], and compounds that work by other mechanisms (for example linomide, inhibitors of integrin αvβ3 function and angiostatin);

(vi) vascular damaging agents such as Combretastatin A4;

(vii) antisense therapies, for example those which are directed to the targets listed above, such as ISIS 2503, an anti-ras antisense;

(viii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and

(ix) immunotherapy approaches, including for example ex-vivo and in-vivo approaches to increase the immunogenecity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

It will be clear to a person skilled in the art that, where appropriate, the other therapeutic ingredient(s) may be used in the form of salts, for example as alkali metal or amine salts or as acid addition salts, or prodrugs, or as esters, for example lower alkyl esters, or as solvates, for example hydrates, to optimise the activity and/or stability and/or physical characteristics, such as solubility, of the therapeutic ingredient. It will be clear also that, where appropriate, the therapeutic ingredients may be used in optically pure form.

When a compound of formula (I) is used in combination with a second therapeutic agent active against the same disease, the dose of each compound may differ from that when the compound is used alone. Appropriate doses will be readily appreciated by those skilled in the art.

The compounds of this invention may be made by a variety of methods, including standard chemistry. Any previously defined variable will continue to have the previously defined meaning unless otherwise indicated. Illustrative general synthetic methods are set out below and then specific compounds of the invention are prepared in the Working Examples.

### General Processes

The present invention also provides processes for the preparation of compounds of formula (I) or salts or solvates thereof. The compounds of this invention may be made by a variety of methods, including standard chemistry. Any previously defined variable will continue to have the previously defined meaning unless otherwise indicated. Illustrative general synthetic methods are set out below and then specific compounds of the invention are prepared in the working Examples.

According to a first process, A, a compound of formula (I) may be prepared by reacting a compound of formula (II)

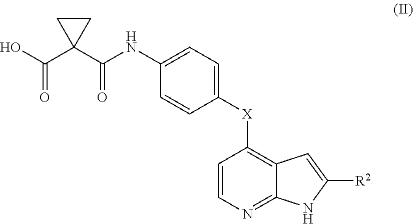

wherein X and R² are as defined hereinabove,
with a compound of formula (III)

H₂N—R¹ (III)

wherein R¹ is as defined hereinabove.

The condensation reaction may typically be carried out in an appropriate solvent, such as N,N-dimethylformamide, dichloromethane, dichloroethane or chloroform with a suitable coupling agent for example, O-benzotriazole-N,N,N′,N′-tetramethyluronium hexafluorophosphate, O-benzotriazole-1-yl-1,1,3,3-tetramethyluronium tetrafluoro borate, 1-hydroxybenzotriazole or O-(7-azabenzotriazol-1-yl)-N,N,N′,N′-tetramethyluronium hexafluorophosphate may be used, optionally with the addition of a suitable base such as diisopropylethylamine. Various temperatures and times may be employed, for example 0 to 60° C. For example, the reaction may be carried out at room temperature for 12 hours. The molar ratio of a compound of formula (II): a compound of formula (III) may be 1:2, for example.

Compounds of formula (II) may be prepared by the methods described herein (see scheme 1) or alternatively may be prepared by methods well known to those skilled in the art.

Compounds of formula (III) are commercially available. For example, 9-aminophenanthrene, 1-aminoanthracene, 1-aminopyrene, 1-napthylamine, 6-aminochrysene, 1-aminoperylene, 1-amino-2-methylnaphthalene, 2-methoxy-5-aminonaphthalene, 8-amino-2-napthol, 1-amino-4-chloronaphthalene, aniline, meta-toluidine, 3-ethylaniline, 3-isopropylaniline, 4-N-butylaniline, 4-pentylaniline, 4-hexylaniline, 4-(2,2,2-trifluoroethyl)aniline, 2-methoxyaniline, para-phenetidine, 4-propoxyaniline, 4-butoxyaniline, 4-pentyloxyaniline, 4-hexyloxyaniline, 2-fluoroaniline, 3-chloroaniline, 4-bromoaniline, 2-iodoaniline, 4-aminophenol, 3-trifluoromethylaniline, 4-(2-bromoethyl)phenylaniline, 4-(2,2,2-trifluoroethyl)aniline, 2-amino-4-chlorophenol, 3,5-bistrifluoromethylaniline, 2,4,6-tribromoaniline, 2,6-dibromo-4-methylaniline, 2,4,6-trimethylaniline are all available from Sigma-Aldrich, Rhintech or Lanxess for example.

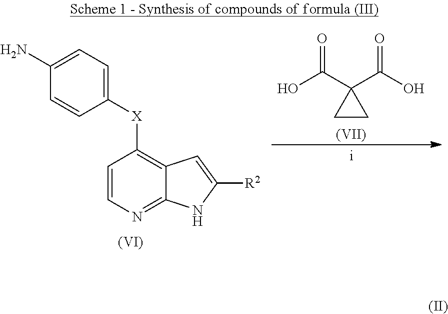

Reagents and conditions: i) Suitable activating agent such as thionyidichloride, POCl₃ or PhPOCl₂, appropriate base such as diisopropylethylamine or triethylamine.

Compounds of formula (VI) may be prepared by methods known to those skilled in the art, or may be prepared by the methods described herein (see schemes 2, 3, 4 and 5).

Compounds of formula (VI) in which X represents O and R² represents hydrogen may be prepared by similar methods to those disclosed by Santiago et al. in Tetrahedron Letters, 47:2069-2072, (2006), see specifically scheme 1, compound 9. Alternatively they may be prepared by methods similar to those disclosed by Thukewohl et al. in Synthesis, 4:629-632, (2006), specifically see example 10 in table 1. Reduction of the nitro group to an amine, using methods well known to those skilled in the art (for example using a reducing agent such as tin, in a solvent such as ethanol and optionally with an acid catalyst such as hydrogen chloride), will yield a compound of formula (VI).

The compound of formula (VII), 1-cyclopropanedicarboxylic acid, is commercially available, for example, from Sigma-Aldrich.

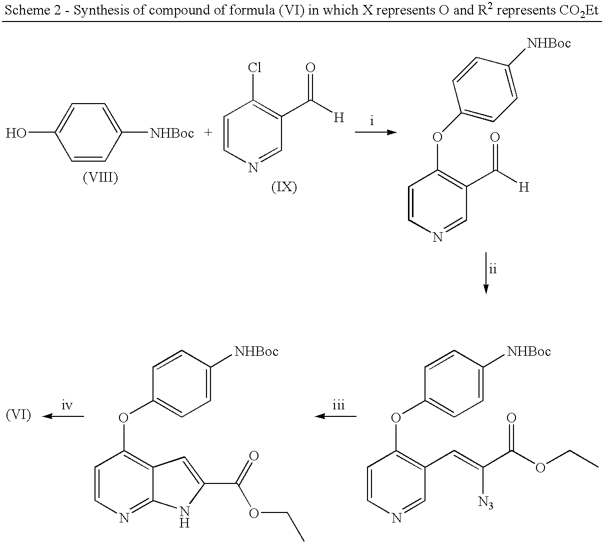

Reagents and conditions: i) Suitable base such as potassium carbonate, in an appropriate solvent such as N,N-dimethylformamide, at an elevated temperature such as 50 to 100° C. e.g. 80° C. for an appropriate time such as 1 hour. ii) Ethyl azidoacetate (commercially available, for example from Apollo or Fluka), in an appropriate solvent such as anhydrous ethanol, followed by addition of a suitable base for example sodium ethoxide in anhydrous ethanol, at a suitable temperature such as −20 to 50° C. e.g. 0° C., for an appropriate time e.g. 1 hour. The ratio of sodium ethoxide to the pyridine derivative may be, for example, 4:1. The ratio of ethyl azidoacetate to the pyridine derivative may also be, for example, 4:1. iii) Suitable elevated temperature such as 100 to 250° C. e.g. 170° C., in a suitable solvent such as o-xylene, for an appropriate time such as 1 hour. iv) Boc deprotection using a suitable acid such as trifluoroacetic acid, in an appropriate solvent such as dichloromethane. The reaction may take place, for example, over 30 minutes at room temperature.

The compound of formula (VIII), 4-N-Boc-aminophenol is commercially available, for example, from Sigma-Aldrich.

The compound of formula (IX) 4-chloro-3-formylpyridine, may be prepared by methods disclosed by Albanese, Pervo and Zenoni in Synthesis, 8:1294-1297, (1999), specifically see compound number 8. Alternatively, they may be prepared by methods well known to those skilled in the art.

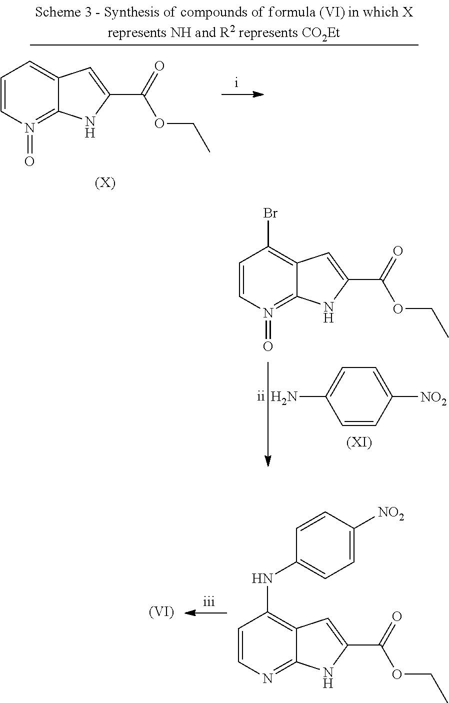

Reagents and Conditions: i) methanesulfonic anhydride, tetramethylammonium bromide, in a suitable solvent such as N,N-dimethylformamide or dimethylsulfoxide, at an appropriate temperature such as approximately 0° C. The molar ratio of the compound of formula (X):tetramethylammonium bromide:methane sulfonic anhydride may be, for example, 1:1.5:2. ii) Suitable solvent such as N,N-dimethylformamide, dioxane, toluene or xylene, with an appropriate base such as caesium carbonate, potassium tert-butoxide or sodium tert-butoxide, suitable palladium-based catalyst such as Pd(dppf)Cl₂, Pd(OAc)₂ or Pd₂(dba)₃. Elevated temperature, for example 60 to 180° C. e.g. 120° C. (optionally using a microwave). The molar ratio of the brominated azaindole derivative:a compound of formula (XI) may be, for example, 1:3. The temperature and length of reaction will vary with solvent system and catalyst, and this will be appreciated by those skilled in the art. iii) Reduction of the nitro group using a suitable reducing agent such as tin, in an appropriate solvent such as ethanol, optionally with an acid catalyst such as hydrogen chloride, and optionally at an elevated temperature such as at reflux.

Compounds of formula (X) may be readily prepared by those skilled in the art using methods disclosed in the international patent application WO 2000/044753, more specifically Example 6, step 3)

The compound of formula (XI), 4-nitroaniline, is commercially available, for example, from Sigma-Aldrich.

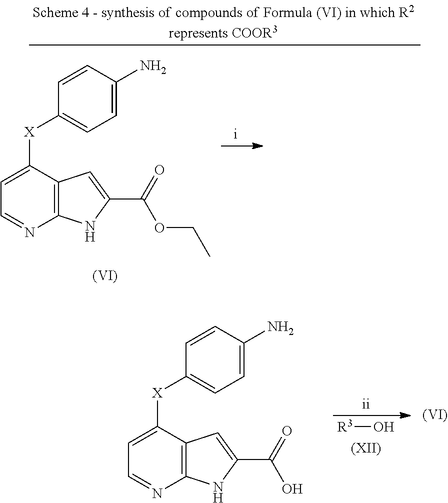

Reagents and conditions: i) Deprotection using an appropriate base such as 6 M sodium hydroxide, in a suitable solvent such as methanol, optionally at an elevated temperature e.g. approximately 60° C. ii) Optionally in a suitable solvent such as acetonitrile, optionally with a suitable catalyst such as dilute hydrogen chloride and optionally at an elevated temperature such as at reflux.

Compounds of formula (XII) are commercially available, for example methanol, ethanol, propanol, butanol, pentanol, hexanol, isopropanol, tert-butanol are all commercially available, for example, from Sigma-Aldrich.

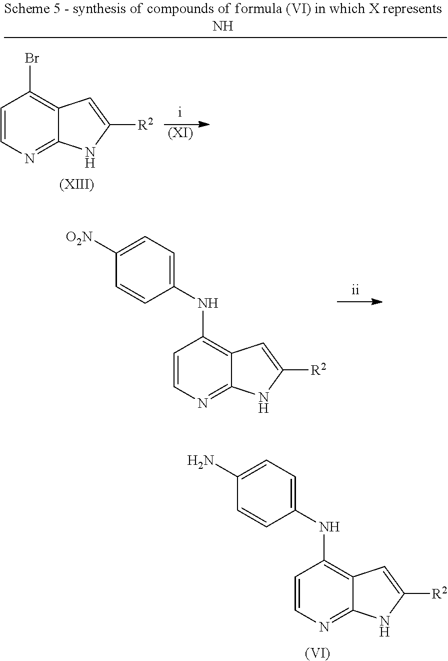

Reagents and Conditions: i) Suitable solvent such as N,N-dimethylformamide, dioxane, toluene or xylene, with an appropriate base such as potassium tert-butoxide caesium carbonate or sodium tert-butoxide, suitable palladium based catalyst such as Pd(dppf)Cl₂, Pd(OAc)₂ or Pd₂(dba)₃, elevated temperature for example 60 to 180° C. e.g. 120° C. (optionally using a microwave). The molar ratio of a compound of formula (XIII):a compound of formula (XI) may be, for example, 1:3. The time and temperature of reaction will vary with the solvent system and catalysts used, but these will be understood by those skilled in the art. ii) Reduction of the nitro group using a suitable reducing agent such as tin, in an appropriate solvent such as ethanol, optionally with an acid catalyst such as hydrogen chloride and optionally at an elevated temperature such as at reflux, for an appropriate length of time, e.g. 30 minutes.

Compounds of formula (XIII) in which R² represents hydrogen may be prepared by methods well-known to those skilled in the art, for example from commercially available 7-ozaindole using the methods disclosed by Thibault et al. in Organic Letters, 5(26):5023-5025, (2003), or by methods disclosed in international patent application WO 2003/000690, more specifically, see Reference Example 18.

Compounds of formula (XIII) in which R² represents aryl may be prepared according to methods described herein (see scheme 7) or by methods well-known to those skilled in the art.

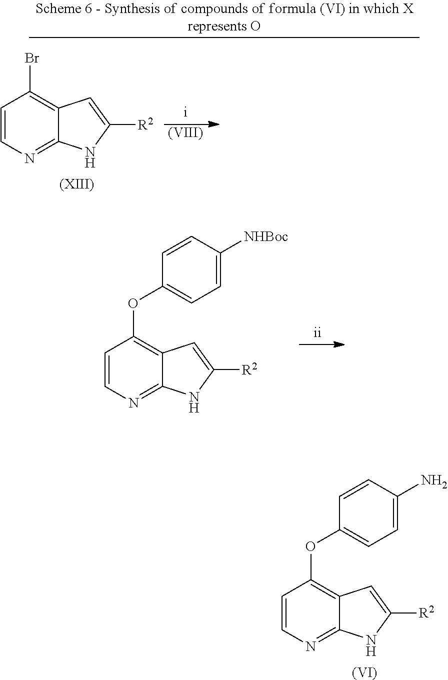

Reagents and conditions: i) Appropriate base such as potassium carbonate, in a suitable solvent such as N,N-dimethylformamide, at an appropriate elevated temperature e.g. approximately 80° C., for a suitable length of time, such as approximately 1 hour. ii) Boc deprotection using a suitable acid such as trifluoroacetic acid, in an appropriate solvent such as dichloromethane. The reaction may take place, for example, at room temperature over 30 minutes.

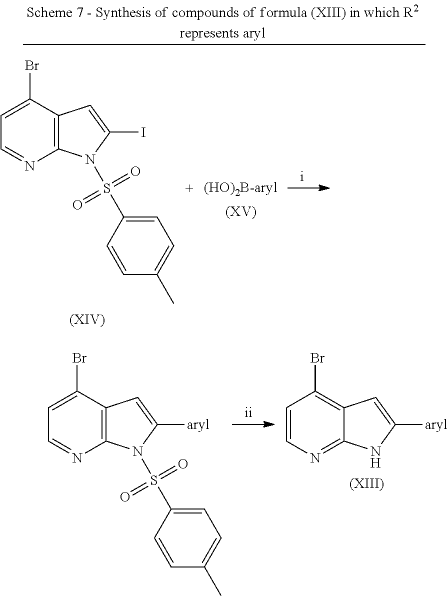

Reagents and Conditions: i) Suitable solvent such as 1,2-dimethoxyethane or N,N-dimethylformamide, dioxane, tetrahydrofuran, methanol, ethanol, acetonitrile etc., in the presence of a suitable palladium catalyst such as Pd(PPh₃)₄ or Pd(OAc)₂ or Pd₂(dba)₃ with an appropriate base such as 2 M aqueous sodium carbonate, potassium carbonate, caesium carbonate, caesium fluoride, sodium hydroxide etc., heated at an elevated temperature such as 60 to 180° C. e.g. 130° C. (optionally using a microwave). Alternatively palladium on carbon or polymer-bound palladium may be used. Times and temperatures will vary depending on the solvent system and catalyst used, but this will be understood by those skilled in the art. The residue may be purified by SCX cartridge in a catch-and-release, for example. ii) Removal of protecting group using an appropriate base such as aqueous sodium hydroxide, in an appropriate solvent such as methanol, and optionally at an elevated temperature such as 70° C., for an appropriate time for example 2 hours.

The compound of formula (XIV) may be prepared according to the methods described in international patent application WO 2003/000690, more specifically, for example, see Reference Example 17. It is to be noted that alternative nitrogen protecting groups may be employed instead of the tosyl group depicted in scheme 7. Such protecting groups are well-known to those skilled in the art.

The boronic acid compounds of formula (XV) are commercially available. For example 1-naphthaleneboronic acid, 6-hydroxy-2-naphthaleneboronic acid, 9-phenanthreneboronic acid, 9-anthraceneboronic acid, phenylboronic acid, 4-fluorophenylboronic acid, 4-bromophenylboronic acid, 3-iodophenylboronic acid, 3-methylphenylboronic acid, 4-propylphenylboronic acid, 4-butylphenylboronic acid, 4-hexylphenylboronic acid, 2,4-dichlorophenylboronic acid, 4-methoxyphenylboronic acid, (4-tertbutoxyphenyl)boronic acid, 4-butoxyphenylboronic acid, 4-hexyloxyphenylboronic acid, 3-hydroxyphenylboronic acid, 4-bromomethylphenylboronic acid, 3,5-bis(trifluoromethyl)benzeneboronic acid, (5-bromo-2-hydroxy)benzeneboronic acid, 2-ethoxy-5-methylphenylboronic acid, 3-chloro-4-fluorophenylboronic acid, 3,5-dimethyl-4-methoxyphenylboronic acid, 2-chloro-4-isopropoxyphenylboronic acid are all commercially available, for example, from Sigma-Aldrich, Fluka, Apollo, Rare Chem and Combi-Blocks Inc.

According to a second process, B, a compound of formula (I) in which R² represents C(O)NR⁴R⁵ may be prepared by reacting a compound of formula (IV)

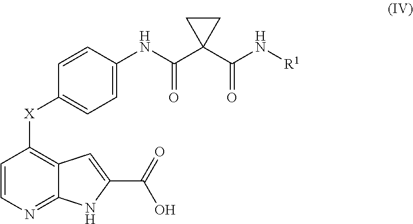

wherein X and R¹ are as defined herein above,
with a compound of formula (V)

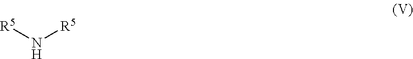

wherein R⁴ and R⁵ are as defined herein above.

The condensation reaction may typically be carried out in a suitable solvent, such as N,N-dimethylformamide. The combination of a suitable activating agent, for example O-benzotriazole-N,N,N′,N′-tetramethyluronium hexafluorophosphate, O-benzotriazole-1-yl-1,1,3,3-tetramethyluronium tetrafluoroborate or 1-hydroxy benzotriazole, with a suitable base such as diisopropylethylamine may be used. Various temperatures may be employed, for example 0 to 60° C. e.g. at room temperature, and times such as approximately 16 hours. Alternative solvents for the reaction include dichloromethane, chloroform or dichloroethane. The molar ratio of a compound of formula (IV):a compound of formula (V) may be, for example, between 1:1 and 1:2, e.g. 1:1.5

After reaction, the product may be isolated by concentration in vacuo and may be purified if required for example by mass-directed autopurification.

Compounds of formula (IV) may be prepared by interconversion of other compounds of formula (I), as depicted in scheme 8.

Compounds of formula (III) are commercially available, or may be prepared by methods well-known to those skilled in the art.

For example, when R⁵ represents —C₁₋₃alkylNR⁶R⁷, examples of suitable commercially available material include, but are not limited to: 1,3-diaminopropane;
1,2-diaminopropane; N-isopropyl-1,3-propanediamine; N-ethylethylenediamine; N—(N-propyl)ethylenediamine, N—N-diethyl-N-methyl-1,3-propanediamine; N,N,N′-trimethyl-1,3-propanediamine; N,N-diethyl-1,3-propanediamine; N-2-aminoethyl homopiperidine; N-(2-aminoethyl)piperazine; N-(2-aminoethyl)morpholine; N-(2-aminoethyl)piperidine; N-(2-aminoethyl)pyrrolidine; 1-methyl-1,2-morpholino-4-yl ethylamine; N,N-diisopropylethylenediamine; N-(3-aminopropyl)morpholine; C-piperidin-1-yl-methylamine; C-morpholin-1-yl-methylamine and 3(-azepanyl)-1-propanamine; all commercially available, for example, from Sigma-Aldrich, Acros, Fluka, Akos, Betapharma or ASDI Inc.

For example, when R⁵ represents —C₁₋₃alkyl-SO₂—C₁₋₃alkyl, examples of suitable compounds of commercially available material include, but are not limited to: [2-(propylsulfonyl)ethyl]amine; [3-(methanesulfonyl)propyl]amine; 2-aminoethylmethyl sulfone and 2-(methylamino)-1-(methylsulfonyl)ethane; all commercially available, for example, from Betapharma, Apollo or Rare Chem.

For example, when R⁵ represents —C₁₋₃alkylOH, examples of suitable commercially available compounds include, but are not limited to: 3-amino-1-propanol; 3-(isopropylamino)-propan-1-ol; 2-(methylamino)ethanol; 2-(ethylamino)ethanol and 2-(propylamino)ethanol; all commercially available, for example, from Sigma-Aldrich or ChemBridge Corp.

For example, when R⁵ represents —C₁₋₃alkyl-C(O)NH₂, examples of suitable commercially available compounds include, but are not limited to: glycinamide, 4-aminobutanamide, β-alaninamide N⁻²-methylglycinamide; all commercially available, for example, from Sigma-Aldrich, AndaChem Inc. or Advanced Chem Tech.

For example, when R⁵ represents —C₁₋₃alkylheteroaryl, examples of suitable commercially available compounds include, but are not limited to: furfurylamine;
thiophene-2-methylamine; N-omega-methyltryptamine; 2-(2H-pyrrole)ethylamine; 2-(aminomethyl)pyridine; 2-(2-methylaminoethyl)pyridine; N-(3-aminopropyl)imidazole; 3-(1H-pyrrol-1-yl)propylamine and 3-(1H-indol-1-yl)propylamine; all commercially available, for example, from Sigma-Aldrich, Fluka, ChemBridge Corp. or Rare Chem.

For example, when R⁵ represents —C₀₋₃alkylaryl, examples of suitable commercially available compounds include, but are not limited to: 1-aminopyrene; 8-amino-2-naphthol; 1-amino-4-bromonaphthalene; 1-aminoanthracene; 9-aminophenanthrene;
N-ethyl-1-naphthylamine; aniline, ortho-toluidine; 2,3-dimethylaniline; 2-isopropylaniline; 3-methoxyaniline; 3-chloroaniline; 2-fluoroaniline; 2,4,6-tribromoaniline; 2-iodoaniline; 3-aminophenol; 3,5-bistrifluoromethylaniline; 2-(4-aminophenyl)ethylamine, 4-(2-methylaminoethyl)phenylamine, N-[3-(4-aminophenyl) propyl]-N,N-dimethylamine, 4-pentylaniline, 4-hexylaniline, (4-butyloxy)aniline, 4-pentyloxyaniline, 4-hexyloxyaniline, 3-methoxy-5-trifluoromethylaniline; N-methylaniline; N-ethylaniline; benzylamine; 2-fluorobenzylamine; N-methylbenzylamine; N-isopropylbenzylamine; 2-phenylethylamine; 1-naphthalenethylamine and 3-phenylpropylamine are all commercially available, for example, from Sigma-Aldrich, Amichem or Apollo.

For example, when R⁵ represents C₃₋₆cycloalkyl, examples of suitable commercially available compounds include, but are not limited to: cyclopropylamine, cyclobutylamine, cyclopropylamine, cyclohexylamine, N-methylcyclohexylamine, N-ethylcyclohexylamine and N-propylcyclohexylamine; all commercially available, for example, from Sigma-Aldrich or ChemBridge Corp.

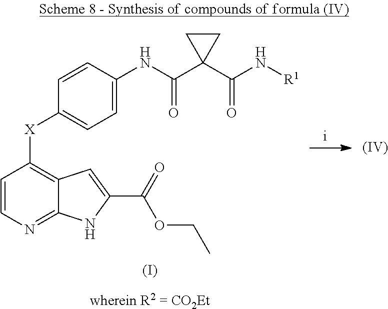

Reagents and conditions: i) Deprotection of acid using an appropriate base, for example 6N sodium hydroxide, in a suitable solvent such as methanol, at an appropriate elevated temperature e.g. approximately 60° C., an appropriate time such as 1 hour.

According to a third process, C, compounds of formula (I) may be prepared by interconversion from other compounds of formula (I).

Interconversions include, but are not limited to alkylation and deprotection, under conditions well-known to those skilled in the art.

Thus typically, an alkylation reaction may be carried out between a compound of formula (I) and a C₁₋₆alkyl, activated to substitution by means of a leaving group such as halogen or an activated hydroxyl group. The reaction takes place in the presence of a suitable base such as NEt₃ or diisopropylethylamine, in an appropriate solvent such as dichloromethane or N,N-dimethylformamide at an appropriate temperature such as about 80° C.

Examples of protecting groups that may be employed in the synthetic routes described and the means for their removal can be found in T. W. Greene ‘Protective Groups in Organic Synthesis’ (3^{rd} edition, J Wiley and Sons, 1999). Suitable amine protecting groups include sulphonyl (e.g. tosyl) acyl (e.g. acetyl, 2′,2′,2′-trichloroethoxycarbonyl, benzyloxycarbonyl or t-butoxycarbonyl) and arylalkyl (e.g. benzyl), which may be removed by hydrolysis (e.g. using an acid such as hydrogen chloride in dioxin or trifluoroacetic acid in dichloromethane) or reductively (e.g. hydrogenolysis of a benzyl group or reductive removal of a 2′,2′,2′-trichloroethoxycarbonyl group using zinc in acetic acid) as appropriate. Other suitable amine protecting groups include trifluoroacetyl (—COCF₃), which may be removed by base catalysed hydrolysis or a solid phase resin bound benzyl group, such as a Merrifield resin bound 2,6-dimethoxybenzyl group (Ellman linker), which may be removed by acid catalysed hydrolysis, for example with trifluoroacetic acid.

According to a fourth process, D, a salt of a compound of formula (I) may be prepared by exchange of counterions, or precipitation of said salt from the free base.

It will be appreciated that all novel intermediates used to prepare compounds of the invention form yet a further aspect of the present invention.

The following examples are intended for illustration only and are not intended to limit the scope of the invention in any way, the invention being defined by the claims which follow. The physical data given for the compounds exemplified is consistent with the assigned structure of those compounds.

### GENERAL EXPERIMENTAL

### Definitions

Unless otherwise noted, all starting materials were obtained from commercial suppliers and used without further purification. Specifically, the following abbreviations may be used in the examples:
BOC (tert-butyloxycarbonyl)
dppf(1,1′-bis(diphenylphosphino)ferrocene);
g (grams);
h (hour);
HEPES (4-(2-hydroxyethyl)-1 piperazine ethane sulfonic acid);
HPLC (High pressure liquid chromatography);
Hz (Hertz);
L (litres);
μL (microlitres);
M (molar);
mg (milligrams);
MHz (megahertz);
min (minutes);
mL (millilitres);
mM (millimolar);
mmol (millimoles);
mol (moles);

All references to ether are to diethyl ether; brine refers to a saturated aqueous solution of NaCl. All reactions are conducted under an inert atmosphere at room temperature unless otherwise noted.

¹H NMR spectra were recorded on a Varian VXR-300, a Varian Unity-300, a Varian Unity-400 instrument, a Brucker AVANCE-400, or a General Electric QE-300. Chemical shifts are expressed in parts per million (ppm, 6 units). Coupling constants are in units of hertz (Hz). Splitting patterns describe apparent multiplicities and are designated as s (singlet), d (doublet), t (triplet), q (quartet), quint (quintet), m (multiplet), br (broad).

Microwave irradiation was performed on a Personal Chemistry Smith Synthesizer™ or Creator™.

SCX purification: Varian Mega Bond Elut SCX; General procedure: A SCX cartridge was rinsed with MeOH, and then crude mixture was dissolved into a suitable solvent such as MeOH, DCM etc. and loaded on the cartridge. And then the cartridge was rinsed with methanol and dichloromethane successively. The product was isolated by elution with a 2M ammonia solution in methanol (for some cases, mixed with DCM), followed by concentration in vacuo.

### General Intermediate 1

### Ethyl 4-[(4-aminophenyl)oxy]-1H-pyrrolo[2,3-b]pyridine-2-carboxylate

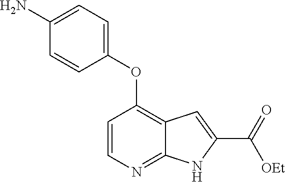

Step A: 1,1-Dimethylethyl{4-[(3-formyl-4-pyridinyl)oxy]phenyl}carbamate

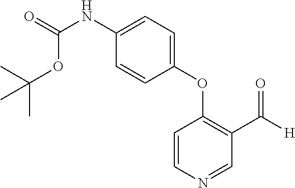

4-Chloro-3-pyridinecarbaldehyde was prepared 4-chloropyridine (commercially available, for example, from Sigma-Aldrich) according to Synthesis 1999, 8, 1294. A mixture of 4-chloro-3-pyridinecarbaldehyde (680 mg, 4.8 mmol), 1,1-dimethylethyl-(4-hydroxyphenyl)carbamate (commercially available, for example, from Sigma-Aldrich) (1 g, 4.8 mmol) and potassium carbonate (1.28 g, 9.4 mmol) in N,N-dimethylformamide was stirred at 80° C. for 1 h. After cooling to room temperature, aqueous ammonium chloride solution was added, and the aqueous layer was extracted with ethyl acetate. The combined organic layer was washed with brine, dried over sodium sulfate, and concentrated under reduced pressure. The resulting solid was collected by filtration, washed with dichloromethane, and dried in vacuo to give the title compound (1.4 g, 93%). ¹H NMR (400 MHz, DMSO-d⁻⁶) ppm 1.49 (s, 9H), 6.71 (d, 1H, J=5.8 Hz), 7.17-7.23 (m, 2H), 7.56-7.62 (m, 2H), 8.58 (d, 1H, J=5.8 Hz), 8.85 (s, 1H), 9.54 (br, 1H), 10.47 (s, 1H).

Step B: Ethyl (2Z)-2-azido-3-(4-{[4-({[(1,1-dimethylethyl)oxy]carbonyl}amino)phenyl]oxy}-3-pyridinyl)-2-propenoate

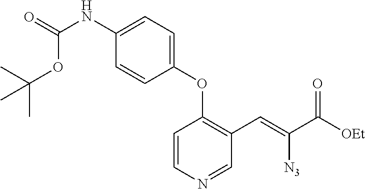

A suspension of 1,1-dImethylethyl{-4-[(3-formyl-4-pyridinyl)oxy]phenyl}carbamate (step A) (1.1 g, 3.5 mmol) and ethyl azidoacetate (commercially available, for example, from Apollo) (1.8 g, 14 mmol) in anhydrous ethanol (20 mL) at 0° C. was added a solution of sodium ethoxide (1.2 g, 14 mmol) in anhydrous ethanol (20 mL) dropwise. Then the mixture was allowed to warm to rt, stirred for 1 h, and quenched with aqueous ammonium chloride solution. The mixture was extracted with ethyl acetate twice, dried over magnesium sulfate, and concentrated in vacuo. The crude product was purified by Yamazen Fast Flow Liquid Chromatography on a silica gel column (dichloromethane/ethyl acetate=9/1 to 3/1) to give the title compound (700 mg, 47%). ¹H NMR (400 MHz, DMSO-d⁻⁶) ppm 1.30 (t, 3H, J=7.1 Hz), 1.48 (s, 9H), 4.33 (q, 2H, J=7.1 Hz), 6.58 (d, 1H, J=5.8 Hz), 7.09-7.14 (m, 2H), 7.16 (s, 1H), 7.53-7.59 (m, 2H), 8.35 (d, 1H, J=5.8 Hz), 9.21 (s, 1H), 9.51 (br, 1H).

Step C: Ethyl 4-[(4-aminophenyl)oxy]-1H-pyrrolo[2,3-b]pyridine-2-carboxylate

A suspension of ethyl (2Z)-2-azido-3-(4-{[4-({[(1,1-dimethylethyl)oxy]carbonyl}amino)phenyl]oxy}-3-pyridinyl)-2-propenoate (Step B) (740 mg, 1.7 mmol) in o-xylene (60 mL) was stirred at 170° C. for 30 min. After cooling to room temperature, o-xylene was removed under reduced pressure, and the resulting solid was washed with ether and dried to give title compound (334 mg). ¹H NMR (400 MHz, DMSO-d⁻⁶) ppm 1.32 (t, 3H, J=7.1 Hz), 1.49 (s, 9H), 4.32 (q, 2H, J=7.1 Hz), 6.40 (d, 1H, J=5.4 Hz), 6.89 (s, 1H), 7.13-7.18 (m, 2H), 7.53-7.59 (m, 2H), 8.24 (d, 1H, J=5.4 Hz), 9.50 (br, 1H), 12.58 (br, 1H).

Trifluoroacetic acid was added to the product and stirred for 30 min. Then trifluoroacetic acid was removed under reduced pressure and the crude solid was purified by SCX cartridge to give the title compound (230 mg, 46%). ¹H NMR (400 MHz, DMSO-d⁻⁶) ppm 1.32 (t, 3H, J=7.1 Hz), 4.32 (q, 2H, J=7.1 Hz), 5.15 (br, 2H), 6.34 (d, 1H, J=5.4 Hz), 6.61-6.67 (m, 2H), 6.87-6.93 (m, 3H), 8.20 (d, 1H, J=5.4 Hz), 12.50 (br, 1H).

### General Intermediate 2

### 4-[(4-{[(1-{[(4-Fluorophenyl)amino]carbonyl}cyclopropyl)carbonyl]amino}phenyl)oxy]-1H-pyrrolo[2,3-b]pyridine-2-carboxylic acid (Example 23)

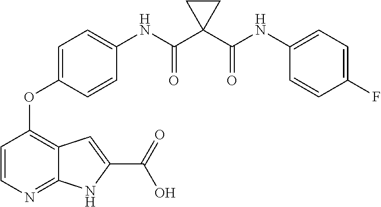

Step A: 1-({[4-([2-[(Ethyloxy)carbonyl]-1H-pyrrolo[2,3-b]pyridin-4-yl]oxy) phenyl]amino}carbonyl)cycloproanecarboxylic acid

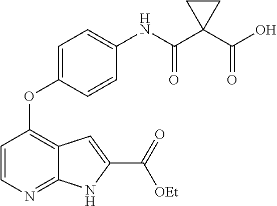

To a stirring solution of 1,1-cyclopropanedicarboxylic acid (commercially available, for example, from Sigma-Aldrich) (31 mg 0.24 mmol) in tetrahydrofuran (0.5 mL) under argon was added triethylamine (34 μL, 0.24 mmol). After 40 min, thionyidichloride (18 μL, 0.24 mmol) was added, and the mixture was stirred at room temperature for 2 h. Then ethyl 4-[(4-aminophenyl)oxy]-1H-pyrrolo[2,3-b]pyridine-2-carboxylate (General Intermediate 1, step C) (61 mg, 0.21 mmol) and tetrahydrofuran (0.5 mL) were added, and the mixture was stirred overnight. The crude mixture was purified by SCX cartridge and Yamazen Fast Flow Liquid Chromatography on a silica gel column (dichloromethane/methanol=1/0 to 4/1) to give the title compound (61 mg). ¹H NMR (400 MHz, DMSO-d⁻⁶) ppm 1.32 (t, 3H, J=7.1 Hz), 1.37 (br, 4H), 4.32 (q, 2H, J=7.1 Hz), 6.43 (d, 1H, J=5.3 Hz), 6.91 (s, 1H), 7.17-7.21 (m, 2H), 7.68-7.74 (m, 2H), 8.25 (d, 1H, J=5.3 Hz), 12.60 (br, 1H).

Step B: 4-[(4-{[(1-{[(4-Fluorophenyl)amino]carbonyl}cyclopropyl)carbonyl]amino}phenyl)oxy]-1H-pyrrolo[2,3-b]pyridine-2-carboxylic acid (Example 24)

To a solution of 1-({[4-({2-[(ethyloxy)carbonyl]-1H-pyrrolo[2,3-b]pyridin-4-yl}oxy)phenyl]amino}carbonyl)cyclopropanecarboxylic acid (Step A) (330 mg, 0.8 mmol) in N,N-dimethylformamide (13 mL) were added o-benzotriazole-1-YL-N,N,N¹,N¹-tetramethyluronium hexafluorophosphate (910 mg, 2.4 mmol), diisopropylethylamine (420 μL, 2.4 mmol), and 4-fluoroaniline (commercially available, for example, from Sigma-Aldrich) (114 μL, 1.2 mmol). The mixture was stirred for 5 h, and then brine was added. The resulting mixture was extracted with ethyl acetate. And the combined organic layer was dried over sodium sulfate, concentrated under reduced pressure, and purified by Yamazen Fast Flow Liquid Chromatography on a silica gel column dichloromethane methanol=1/0 to 9/1) to give the title compound (205 mg). ¹H NMR (400 M Hz, DMSO-d⁻⁶) ppm 1.32 (t, 3H, J=7.2 Hz), 1.47 (s, 4H), 4.32 (q, 2H, J=7.2 Hz), 6.43 (d, 1H, J=5.6 Hz), 6.92 (s, 1H), 7.11-7.24 (m, 4H), 7.57-7.69 (m, 2H), 7.69-7.80 (m, 2H), 8.26 (d, 1H, J=5.6 Hz), 10.06 (br, 1H), 10.17 (br, 1H), 12.61 (br, 1H).

The product was dissolved in methanol, 6 N aqueous sodium hydroxide solution was added, and the mixture was stirred at 60° C. for 1 h. After cooling to room temperature, the mixture was acidified with 2 N hydrogen chloride and purified by SCX cartridge to give the title compound. ¹H NMR (400 MHz, DMSO-d⁻⁶) ppm 1.39-1.53 (m, 4H), 6.05 (s, 1H), 6.44 (d, 1H, J=5.4 Hz), 7.07-7.19 (m, 4H), 7.62-7.74 (m, 4H), 8.02 (d, 1H, J=5.4 Hz), 10.06 (br, 1H), 10.15 (br, 1H), 11.12 (br, 1H).

### Example 1

### N¹-(4-Fluorophenyl)-N¹-(4-{[2-({[2-(methylsulfonyl)ethyl]amino}carbonyl)-1H-pyrrolo[2,3-b]pyridin-4-yl]oxy}phenyl)-1,1-cyclopropane dicarboxamide

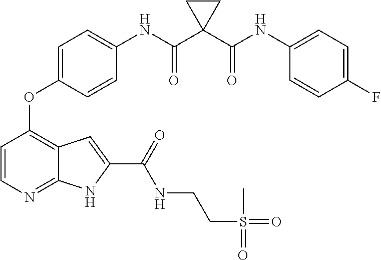

To a solution of 4-[(4-{[(1-{[(4-fluorophenyl)amino]carbonyl}cyclopropyl)carbonyl]amino}phenyl)oxy]-1H-pyrrolo[2,3-b]pyridine-2-carboxylic acid (General Intermediate 2) (25 mg, 0.05 mmol) in N,N-dimethylformamide (0.5 mL) were added O-benzotriazole-1-YL-N,N,N¹,N¹-tetramethyluronium hexafluorophosphate (50 mg, 0.13 mmol), diisopropylethamine (26 μL, 0.15 mmol), 2-(methylsulfonyl)ethanamine (commercially available, for example, from BetaPharma) (9 mg, 0.075 mmol). The mixture was stirred at room temperature for 16 hours. N,N-dimethylformamide was removed under reduced pressure, and purified by LC/MS. ¹H NMR (400 MHz, DMSO-d⁶) ppm 1.46 (s, 4H), 3.04 (s, 3H), 3.39 (dd, 2H, J=7.1, 6.6 Hz), 3.69 (dd, 2H, J=12.4, 6.3 Hz), 6.37 (d, 1H, J=5.4 Hz), 7.09 (s, 1H), 7.11-7.23 (m, 4H), 7.58-7.68 (m, 2H), 7.68-7.79 (m, 2H), 8.18 (d, 1H, J=5.4 Hz), 8.76 (t, 1H, J=5.2 Hz), 10.05 (br, 1H), 10.16 (br, 1H), 12.28 (br, 1H).

### Example 2

### N¹-(4-{[2-({[2-(Dimethylamino)ethyl]amino}carbonyl)-1H-pyrrolo[2,3-b]pyridin-4-yl]oxy}phenyl)-N¹-(4-fluorophenyl)-1,1-cyclopropanedicarboxamide

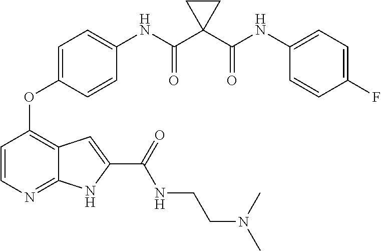

A similar procedure as Example 1 was used, with N,N-dimethyl-1,2-ethanediamine (commercially available, for example from Sigma-Aldrich) being substituted for 2-(methylsulfonyl)ethanamine, to prepare the title compound. ¹H NMR (400 MHz, DMSO-d⁶) ppm 1.46 (s, 4H), 2.18 (s, 6H), 2.40 (dd, 2H, J=6.6, 6.8 Hz), 6.37 (d, 1H, J=5.6 Hz), 7.06 (s, 1H), 7.13-7.22 (m, 4H), 7.60-7.67 (m, 2H), 7.69-7.77 (m, 2H), 8.17 (d, 1H, J=5.6 Hz), 8.40 (t, 1H, J=5.7 Hz), 10.06 (br, 1H), 10.15 (br, 1H).

### Example 3

### N¹-(4-Fluorophenyl)-N¹-(4-{[2-({[2-(1-piperazinyl)ethyl]amino}carbonyl)-1H-pyrrolo[2,3-b]pyridin-4-yl]oxy}phenyl)-1,1-cyclopropane dicarboxamide

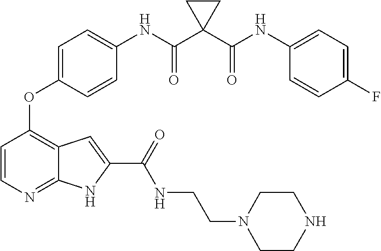

A similar procedure as Example 1 was used, with 1,1-dimethylethyl 4-(2-aminoethyl)-1-piperazinecarboxylate (commercially available, for example, from Sigma-Aldrich) being substituted for 2-(methylsulfonyl)ethanamine, followed by Boc-deprotection using trifluoroacetic acid to prepare the title compound. ¹H NMR (400 MHz, DMSO-d⁶) ppm 1.47 (s, 4H), 2.32-2.40 (m, 4H), 2.43 (dd, 2H, J=6.8, 6.8 Hz), 2.70 (dd, 4H, J=4.8, 4.8 Hz), 6.37 (d, 1H, J=5.3 Hz), 7.06 (s, 1H), 7.11-7.21 (m, 4H), 7.59-7.68 (m, 2H), 7.69-7.77 (m, 2H), 8.17 (d, 1H, J=5.3 Hz), 8.39 (t, 1H, J=5.7 Hz), 10.11 (br, 2H), 12.20 (br, 1H).

### Example 4

### N¹-(4-Fluorophenyl)-N¹-(4-{[2-({[2-(4-morpholinyl)ethyl]amino}carbonyl)-1H-pyrrolo[2,3-b]pyridin-4-yl]oxy}phenyl)-1,1-cyclopropane dicarboxamide

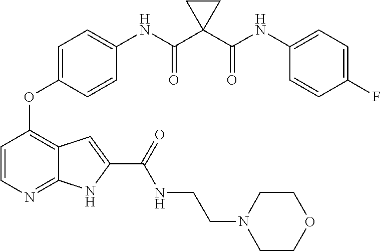

A similar procedure as Example 1 was used, with 2-(4-morpholinyl)ethanamine (commercially available, for example, from Sigma-Aldrich) being substituted for 2-(methylsulfonyl)ethanamine (, to prepare the title compound. ¹H NMR (400 MHz, DMSO-d⁶) ppm 1.46 (s, 4H), 2.38-2.49 (m, 6H), 3.40 (s, 2H), 3.57 (dd, 4H, J=4.8, 4.6 Hz), 6.37 (d, 1H, J=5.3 Hz), 7.07 (s, 1H), 7.10-7.21 (m, 4H), 7.60-7.68 (m, 2H), 7.68-7.77 (m, 2H), 8.17 (d, 1H, J=5.3 Hz), 8.42 (t, 1H, J=5.7 Hz), 10.06 (br, 1H), 10.16 (br, 1H), 12.21 (br, 1H).

### Example 5

### N¹-(4-{[2-({[3-(Dimethylamino)propyl]amino}carbonyl)-1H-pyrrolo[2,3-b]pyridin-4-yl]oxy}phenyl)-N¹-(4-fluorophenyl)-1,1-cyclopropane dicarboxamide

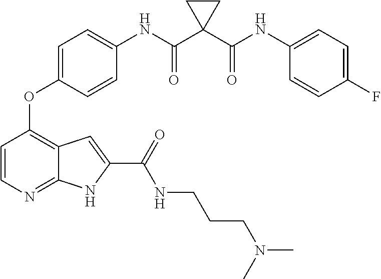

A similar procedure as Example 1 was used, with N,N-dimethyl-1,3-propanediamine

(commercially available, for example, from Sigma-Aldrich) being substituted for 2-(methylsulfonyl)ethanamine, to prepare the title compound. ¹H NMR (400 MHz, DMSO-d⁶) ppm 1.46 (s, 4H), 1.66 (ddd, 2H, J=14.1, 7.2, 6.8 Hz), 2.14 (s, 6H), 2.27 (dd, 2H, J=7.2, 7.2 Hz), 6.37 (d, 1H, J=5.6 Hz), 7.05 (s, 1H), 7.12-7.19 (m, 4H), 7.61-7.67 (m, 2H), 7.69-7.75 (m, 2H), 8.17 (d, 1H, J=5.6 Hz), 8.48 (t, 1H, J=5.6 Hz), 10.06 (br, 1H), 10.16 (br, 1H), 12.17 (br, 1H).

### Example 6

### N¹-{4-[(2-{[[2-(Dimethylamino)ethyl](methyl)amino]carbonyl}-1H-pyrrolo[2,3-b]pyridin-4-yl)oxy]phenyl}-N¹-(4-fluorophenyl)-1,1-cyclopropane dicarboxamide

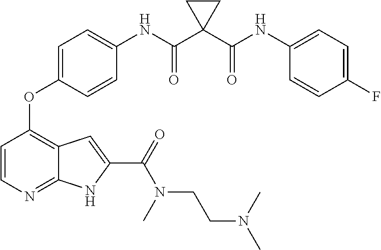

A similar procedure as Example 1 was used, with N,N,N′-trimethyl-1,2-ethanediamine (commercially available, for example, from Sigma-Aldrich) being substituted for 2-(methylsulfonyl)ethanamine, to prepare the title compound. ¹H NMR (400 MHz, DMSO-d⁶) ppm 1.47 (s, 4H), 2.16 (s, 6H), 2.46 (dd, 2H, J=6.6, 6.3 Hz), 3.10 (br, 3H), 3.57 (dd, 2H, J=6.3, 6.1 Hz), 6.33-6.71 (m, 2H), 7.12-7.22 (m, 4H), 7.61-7.67 (m, 2H), 7.69-7.75 (m, 2H), 8.16 (d, 1H, J=5.6 Hz), 10.07 (br, 1H), 10.16 (br, 1H), 12.46 (br, 1H).

### Example 7

### N¹-(4-Fluorophenyl)-N¹-{4-[(2-{[(2-hydroxyethyl)amino]carbonyl}-1H-pyrrolo[2,3-b]pyridin-4-yl)oxy]phenyl}-1,1-cyclopropanedicarboxamide

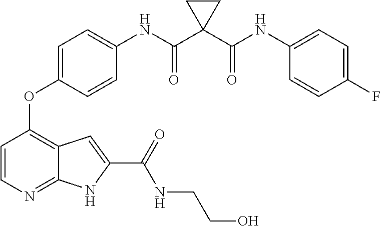

A similar procedure as Example 1 was used, with 2-aminoethanol (commercially available, for example, from Sigma-Aldrich) being substituted for 2-(methylsulfonyl)ethanamine, to prepare the title compound. ¹H NMR (400 MHz, DMSO-d⁶) ppm 1.46 (s, 4H), 3.49-3.55 (m, 2H), 4.79 (dd, 1H, J=5.3, 5.6 Hz), 6.38 (d, 1H, J=5.4 Hz), 7.08 (s, 1H), 7.11-7.20 (m, 4H), 7.60-7.67 (m, 2H), 7.69-7.76 (m, 2H), 8.17 (d, 1H, J=5.4 Hz), 8.49 (t, 1H, J=5.6 Hz), 10.08 (br, 1H), 10.16 (br, 1H), 12.18 (br, 1H).

### Example 8

### N¹-{4-[(2-{[(2-Amino-2-oxoethyl)amino]carbonyl}-1H-pyrrolo[2,3-b]pyridin-4-yl)oxy]phenyl}-N¹-(4-fluorophenyl)-1,1-cyclopropanedicarboxamide

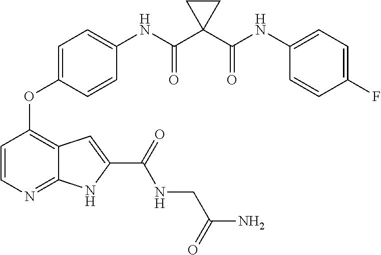

A similar procedure as Example 1 was used, with glycinamide (commercially available, for example, from Sigma-Aldrich) being substituted for 2-(methylsulfonyl)ethanamine, to prepare the title compound. ¹H NMR (400 MHz, DMSO-d⁶) ppm 1.47 (s, 4H), 3.84 (d, 2H, J=6.0 Hz), 6.39 (d, 1H, J=5.6 Hz), 7.08 (br, 1H), 7.09 (s, 1H), 7.12-7.20 (m, 4H), 7.42 (br, 1H), 7.61-7.67 (m, 2H), 7.70-7.75 (m, 2H), 8.18 (d, 1H, J=5.6 Hz), 8.71 (t, 1H, J=6.0 Hz), 10.07 (br, 1H), 10.16 (br, 1H), 12.24 (br, 1H). MS (ESI): m/z 529 (M−1)−, 531 (M+1)+

### Example 9

### N¹-(4-Fluorophenyl)-N¹-(4-{[2-({[2-(1H-imidazol-4-yl)ethyl]amino}carbonyl)-1H-pyrrolo[2,3-b]pyridin-4-yl]oxy}phenyl)-1,1-cyclopropane dicarboxamide

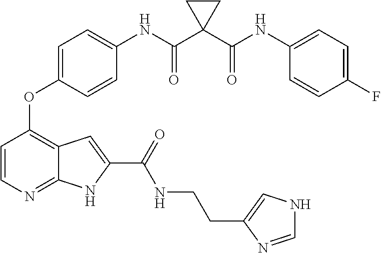

A similar procedure as Example 1 was used, with [2-(1H-imidazol-4-yl)ethyl]amine

(commercially available, for example, from Sigma-Aldrich) being substituted for 2-(methylsulfonyl)ethanamine, (commercially available, for example, from Sigma-Aldrich) to prepare the title compound. ¹H NMR (400 MHz, DMSO-d⁶) ppm 1.47 (s, 4H), 2.76 (dd, 2H, J=7.3, 7.1 Hz), 3.46-3.53 (m, 2H), 6.36 (d, 1H, J=5.4 Hz), 6.83 (br, 1H), 7.06 (s, 1H), 7.12-7.20 (m, 4H), 7.53 (s, 1H), 7.61-7.67 (m, 2H), 7.69-7.75 (m, 2H), 8.17 (d, 1H, J=5.4 Hz), 8.56 (t, 1H, J=5.6 Hz), 10.07 (br, 1H), 10.16 (br, 1H), 12.19 (br, 1H).

### Example 10

### N¹-(4-Fluorophenyl)-N¹-[4-({2-[({2-[3-hydroxy-4-(methyloxy)phenyl]ethyl}amino)carbonyl]-1H-pyrrolo[2,3-b]pyridin-4-yl}oxy)phenyl]-1,1-cyclopropanedicarboxamide

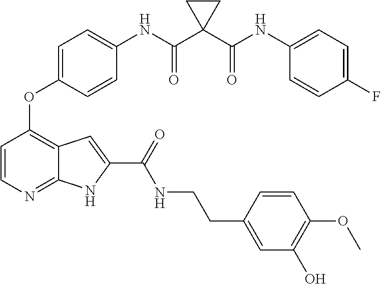

A similar procedure as Example 1 was used, with 5-(2-aminoethyl)-2-(methyloxy)phenol (commercially available, for example, from Sigma Aldrich) being substituted for 2-(methylsulfonyl)ethanamine, to prepare the title compound. ¹H NMR (400 MHz, DMSO-d⁶) ppm 1.46 (s, 4H), 2.70 (dd, 2H, J=7.6, 7.3 Hz), 3.39-3.47 (m, 2H), 3.72 (s, 3H), 6.36 (d, 1H, J=5.3 Hz), 6.61 (dd, 1H, J=8.2, 2.0 Hz), 6.67 (d, 1H, J=2.0 Hz), 6.82 (d, 1H, J=8.2 Hz), 7.06 (s, 1H), 7.12-7.20 (m, 4H), 7.61-7.67 (m, 2H), 7.69-7.75 (m, 2H), 8.16 (d, 1H, J=5.3 Hz), 8.53 (t, 1H, J=5.8 Hz), 8.87 (br, 1H), 10.06 (br, 1H), 10.16 (br, 1H), 12.18 (br, 1H).

### Example 11

### N¹-[4-({2-[({[4-(Dimethylamino)phenyl]methyl}amino)carbonyl]-1H-pyrrolo[2,3-b]pyridin-4-yl}oxy)phenyl]-N¹-(4-fluorophenyl)-1,1-cyclopropane dicarboxamide

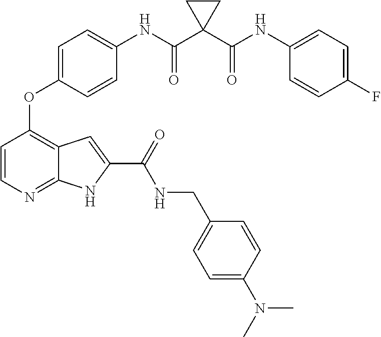

A similar procedure as Example 1 was used, with 4-(aminomethyl)-N,N-dimethylaniline (commercially available, for example, from Sigma-Aldrich) being substituted for 2-(methylsulfonyl)ethanamine, to prepare the title compound. ¹H NMR (400 MHz, DMSO-d⁶) ppm 1.46 (s, 4H), 2.85 (s, 6H), 4.36 (d, 2H, J=5.8 Hz), 6.37 (d, 1H, J=5.6 Hz), 6.67-6.72 (m, 2H), 7.11 (s, 1H), 7.12-7.19 (m, 6H), 7.61-7.66 (m, 2H), 7.68-7.76 (m, 2H), 8.16 (d, 1H, J=5.6 Hz), 8.88 (t, 1H, J=5.8 Hz), 10.06 (s, 1H), 10.15 (br, 1H), 12.20 (br, 1H).

### Example 12

### N¹-(4-Fluorophenyl)-N¹-[4-({2-[({[2-(methyloxy)phenyl]methyl}amino)carbonyl]-1H-pyrrolo[2,3-b]pyridin-4-yl}oxy)phenyl]-1,1-cyclopropane dicarboxamide

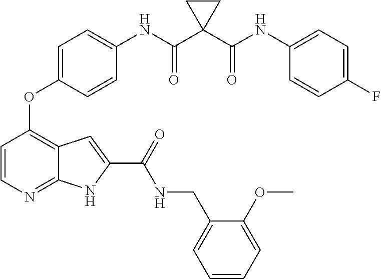

A similar procedure as Example 1 was used, with 1-[2-(methyloxy)phenyl]methanamine (commercially available, for example, from being substituted for 2-(methylsulfonyl)ethanamine, to prepare the title compound. ¹H NMR (400 MHz, DMSO-d⁶) ppm 1.46 (s, 4H), 3.83 (s, 3H), 4.46 (d, 2H, J=5.8 Hz), 6.37 (d, 1H, J=5.6 Hz), 6.89-6.94 (m, 1H), 6.98-7.03 (m, 1H), 7.12-7.28 (m, 7H), 7.61-7.67 (m, 2H), 7.69-7.75 (m, 2H), 8.18 (d, 1H, J=5.6 Hz), 8.84 (t, 1H, J=5.8 Hz), 10.06 (br, 1H), 10.16 (br, 1H), 12.24 (br, 1H).

### Example 13

### N¹-(4-Fluorophenyl)-N¹-(4-{[2-({[2-(methyloxy)phenyl]amino}carbonyl)-1H-pyrrolo[2,3-b]pyridin-4-yl]oxy}phenyl)-1,1-cyclopropane dicarboxamide

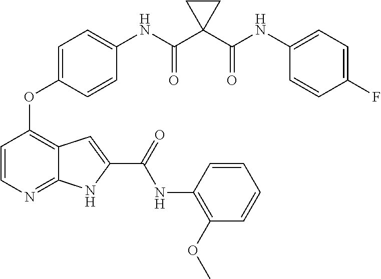

A similar procedure as Example 1 was used, with 2-(methyloxy)aniline (commercially available, for example, from Sigma-Aldrich) being substituted for 2-(methylsulfonyl)ethanamine, to prepare the title compound. ¹H NMR (400 MHz, DMSO-d⁶) ppm 1.47 (s, 4H), 3.85 (s, 3H), 6.38 (d, 1H, J=5.3 Hz), 6.96-7.01 (m, 1H), 7.08-7.24 (m, 6H), 7.28 (br, 1H), 7.61-7.67 (m, 2H), 7.71-7.82 (m, 3H), 8.21 (d, 1H, J=5.3 Hz), 9.49 (br, 1H), 10.06 (br, 1H), 10.17 (br, 1H), 12.46 (br, 1H).

### Example 14

### N¹-[4-({2-[({3-[(Dimethylamino)methyl]phenyl}amino)carbonyl]-1H-pyrrolo[2,3-b]pyridin-4-yl}oxy)phenyl]-N¹-(4-fluorophenyl)-1,1-cyclopropane dicarboxamide

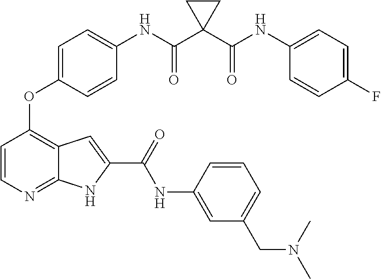

A similar procedure as Example 1 was used, with 3-[(dimethylamino)methyl]aniline (commercially available, for example from J & W PharmLab) being substituted for 2-(methylsulfonyl)ethanamine, to prepare the title compound. ¹H NMR (400 MHz, DMSO-d⁶) ppm 1.47 (s, 4H), 2.16 (s, 6H), 3.38 (s, 2H), 6.38 (d, 1H, J=5.3 Hz), 6.99-7.04 (m, 1H), 7.12-7.24 (m, 4H), 7.27-7.33 (m, 1H), 7.46 (s, 1H), 7.61-7.67 (m, 2H), 7.71-7.77 (m, 4H), 8.22 (d, 1H, J=5.3 Hz), 10.07 (br, 1H), 10.17 (br, 1H), 10.21 (br, 1H), 12.38 (br, 1H).

### Example 15

### N¹-(4-Fluorophenyl)-N¹-[4-(1H-pyrrolo[2,3-b]pyridin-4-ylamino) phenyl]-1,1-cyclopropanedicarboxamide

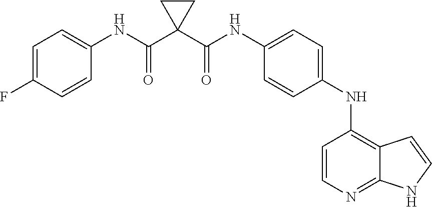

Step A: N-(4-Nitrophenyl)-1H-pyrrolo[2,3-b]pyridin-4-amine

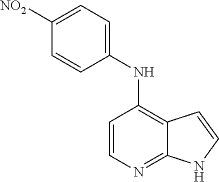

4-Bromo-1H-pyrrolo[2,3-b]pyridine was prepared from commercially available 7-azaindole according to the methods described in Org. Lett., 2003, 5, 26, 5023. To a suspension of 4-bromo-1H-pyrrolo[2,3-b]pyridine (493 mg, 2.5 mmol), potassium tert-butoxide (561 mg, 5.0 mmol) and 4-nitroaniline (commercially available, for example, from Sigma-Aldrich) (1 g, 7.5 mmol) in N,N-dimethylformamide (18 mL) in a microwave vial was added Pd(dppf)Cl₂ (204 mg, 0.3 mmol). After capping, the mixture was heated with Creator™ at 120° C. for 1.5 h. After cooling down, saturated aqueous ammonium chloride was added and the resulting mixture was extracted with dichloromethane (20 mL, 3 times). The combined organic layer was washed with brine, dried over sodium sulfate, and then evaporated to dryness under reduced pressure. The residue was purified by Yamazen Fast Flow Liquid Chromatography on a silica gel column (dichloromethane/methanol=1/0 to 9/1), to give the title compound (536.4 mg, 42%). ¹H NMR (400 MHz, DMSO-d⁶) ppm 6.53 (dd, J=1.9, 3.4 Hz, 1H), 6.98 (d, J=5.3 Hz, 1H), 7.30-7.39 (m, 3H), 8.10 (d, J=5.3 Hz, 1H), 8.18 (d, J=9.1 Hz, 2H), 9.55 (s, 1H), 11.62 (brs, 1H).

Step B: N-1H-Pyrrolo[2,3-b]pyridin-4-yl-1,4-benzenediamine

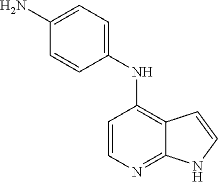

Aqueous hydrogen chloride (2 N, 1 mL) was added to a suspension of N-(4-nitrophenyl)-1H-pyrrolo[2,3-b]pyridin-4-amine (Step A) (300 mg, 1.2 mmol) and tin (700 mg, 5.9 mmol) in ethanol (100 mL). The mixture was stirred at reflux for 30 min, and directly charged into SCX cartridge. After SCX purification, the residue was purified by Yamazen Fast Flow Liquid Chromatography on a silica gel column dichloromethane/methanol=1/0 TO 9/1) to give the corresponding product without the calculation of the yield. ¹H NMR (400 MHz, DMSO-d⁶) ppm 4.98 (brs, 2H), 6.24 (d, J=5.6 Hz, 1H), 6.52 (dd, J=1.8, 3.3 Hz, 1H), 6.60 (d, J=8.6 Hz, 2H), 6.95 (d, J=8.6 Hz, 2H), 7.08 (dd, J=2.3, 3.3 Hz, 1H), 7.75 (d, J=5.6 Hz, 1H), 8.13 (s, 1H), 11.15 (brs, 1H).

Step C: 1-({[4-(1H-Pyrrolo[2,3-b]pyridin-4-ylamino)phenyl]amino}carbonyl)cyclopropanecarboxylic acid

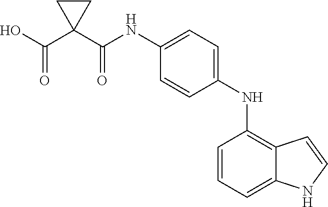

To a stirring solution of 1,1-cyclopropanedicarboxylic acid (commercially available, for example, from Sigma-Aldrich) (65.1 mg 0.5 mmol) in tetrahydrofuran (5 mL) under argon was added triethylamine (50.6 mg, 0.5 mmol). After stirring 30 min at 0° C., thionyldichloride (59.5 mg, 0.5 mmol) was added. The mixture was warmed to room temperature and stirred for another 20 min. The resulting solution (3 mL) was added to a solution of N-1H-pyrrolo[2,3-b]pyridin-4-yl-1,4-benzenediamine (Step B) (67.3 mg, 0.3 mmol) in tetrahydrofuran (1.0 mL), and the mixture was stirred overnight. After evaporating the solvent in vacuo, the residue was purified via recrystallization with methanol and dichloromethane to obtain the title compound (37.0 mg, 37%). ¹H NMR (400 MHz, DMSO-d⁶) ppm 1.39 (s, 4H), 6.57 (d, J=5.6 Hz, 1H), 6.60 (dd, J=1.6, 3.4 Hz, 1H), 7.18 (dd, J=2.1, 3.4 Hz, 1H), 7.23 (d, J=8.8 Hz, 2H), 7.60 (d, J=8.8 Hz, 2H), 7.87 (d, J=5.6 Hz, 1H), 8.67 (s, 1H), 11.08 (brs, 1H), 11.39 (s, 1H), 13.14 (brs, 1H).

Step D: N¹-(4-Fluorophenyl)-N¹-[4-(1H-pyrrolo[2,3-b]pyridin-4-ylamino)phenyl]-1,1-cyclopropanedicarboxamide (title compound)

To a solution of 1-({[4-(1H-pyrrolo[2,3-b]pyridin-4-ylamino)phenyl]amino}carbonyl)cyclopropanecarboxylic acid (Step C) (20 mg, 0.06 mmol) in N,N-Dimethylformamide (2 mL) were added o-Benzotriazole-1-YL-N,N, N¹,N′-tetramethyluronium hexafluorophosphate (34.1 mg, 0.09 mmol) and 4-fluoroaniline (commercially available, for example, from Sigma-Aldrich) (13.3 mg, 0.12 mmol). The mixture was stirred overnight, and directly charged into SCX cartridge. After SCX purification, the residue was purified by Yamazen Fast Flow Liquid Chromatography on a silica gel column dichloromethane/methanol=1 0 to 9 1) to afford the corresponding product without the calculation of the yield. ¹H NMR (400 MHz, DMSO-d⁶) ppm 1.46 (s, 4H), 6.55-6.60 (m, 2H), 7.11-7.18 (m, 3H), 7.22 (d, J=9.1 Hz, 2H), 7.59 (d, J=9.1 Hz, 2H), 7.61-7.66 (m, 2H), 7.86 (d, J=5.6 Hz, 1H), 8.52 (s, 1H), 10.00 (s, 1H), 10.10 (s, 1H), 11.29 (brs, 1H).

### Example 16

### N¹-Phenyl-N¹-[4-(1H-pyrrolo[2,3-b]pyridin-4-ylamino)phenyl]-1,1-cyclopropanedicarboxamide

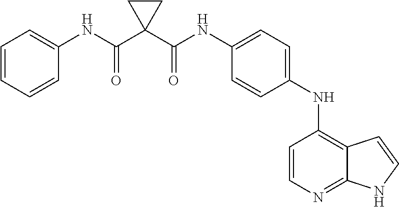

A similar procedure as Example 15 was used, with aniline (commercially available, for example, from Sigma-Aldrich) being substituted for 4-fluoroaniline in Step D, to prepare the title compound. ¹H NMR (400 MHz, DMSO-d⁶) ppm 1.48 (d, J=1.5 Hz, 4H), 6.56-6.59 (m, 2H), 7.04-7.10 (m, 1H), 7.17 (dd, J=2.5, 3.3 Hz, 1H), 7.23 (d, J=8.8 Hz, 2H), 7.31 (dd, J=7.6, 8.3 Hz, 2H), 7.56-7.65 (m, 4H), 7.86 (d, J=5.6 Hz, 1H), 8.53 (s, 1H), 9.96 (s, 1H), 10.11 (s, 1H), 11.29 (brs, 1H).

### Example 17

### Ethyl 4-[(4-{[(1-{[(4-fluorophenyl)amino]carbonyl}cyclopropyl) carbonyl]amino}phenyl)amino]-1H-pyrrolo[2,3-b]pyridine-2-carboxylate

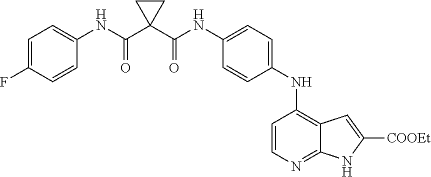

Step A: Ethyl 4-bromo-1H-pyrrolo[2,3-b]pyridine-2-carboxylate

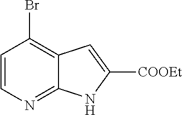

Ethyl 1H-pyrrolo[2,3-b]pyridine-2-carboxylate-7-oxide (1 g, 5 mmol), (prepared using methods disclosed in WO 2000/044753 see Example 6, Step C), was added to a suspension of tetramethylammonium bromide (1.2 g, 7.5 mmol) in N,N-dimethylformamide (50 mL). The resulting mixture was cooled to 0° C. and methanesulfonic anhydride (1.7 g, 10 mmol) was added portionwise. After being warmed up to room temperature and stirred for another 6 hours, the reaction mixture was poured into water (100 mL). After neutralization with 50% aqueous sodium hydroxide, the resulting solution was extracted with ethyl acetate, followed by washing the organic layer with brine and water. Concentration in vacuo gave the residue, which underwent SCX purification to afford the title compound as a pale yellow solid (1 g, 77%). ¹H NMR (400 MHz, DMSO-d⁶) ppm 1.35 (t, 3H, J=7.1 Hz), 4.36 (q, 2H, J=7.1 Hz), 7.05 (d, 1H, J=2.0 Hz), 7.48 (d, 1H, J=5.1 Hz), 8.28 (d, 1H, J=5.1 Hz), 12.95 (brs, 1H).

Step B: Ethyl 4-[(4-nitrophenyl)amino]-1H-pyrrolo[2,3-b]pyridine-2-carboxylate

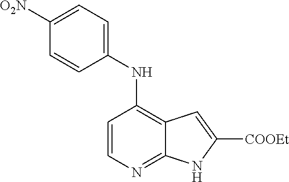

To a suspention of ethyl 4-bromo-1H-pyrrolo[2,3-b]pyridine-2-carboxylate (Step A) (538.2 mg, 2 mmol), potassium tert-butoxide (448.8 mg, 4 mmol), 4-nitroaniline (commercially available, for example, from Sigma-Aldrich) (828.8 mg, 6 mmol), and N,N-dimethylformamide (18 mL) in a microwave vial was added Pd(dppf)Cl₂ (326.6 mg, 0.4 mmol). After capping, the mixture was heated with Creator™ at 120° C. for 30 min. The reaction mixture was quenched by saturated aqueous ammonium chloride, and extracted with dichloromethane (20 mL, 3 times). The organic layer was washed with brine, dried over sodium sulfate, and then evaporated to dryness under reduced pressure. The residue was used for the next step without further purification.

Step C: Ethyl 4-[(4-aminophenyl)amino]-1H-pyrrolo[2,3-b]pyridine-2-carboxylate

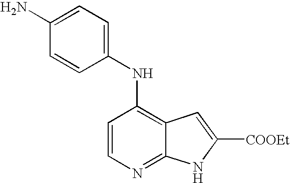

Aqueous hydrogen chloride (6 N, 1 mL) was added to a suspension of the above residue from Step B and tin (1.19 g, 10 mmol) in ethanol (100 mL). The mixture was stirred at 80° C. for 60 min, and directly charged into SCX cartridge. After SCX purification, the residue was purified by Yamazen Fast Flow Liquid Chromatography on a silica gel column dichloromethane/methanol=1/0 TO 9/1) to give the corresponding product (204.9 mg, Total 35% [Step B and C]). ¹H NMR (400 MHz, DMSO-d⁶) ppm 1.32 (t, J=7.2 Hz, 3H), 4.30 (q, J=7.2 Hz, 2H), 5.04 (s, 2H), 6.26 (d, J=5.6 Hz, 1H), 6.61 (d, J=8.6 Hz, 2H), 6.96 (d, J=8.6 Hz, 2H), 7.40 (s, 1H), 7.90 (d, J=5.6 Hz, 1H), 8.53 (s, 1H), 11.98 (s, 1H).

Step D: 1-({[4-({2-[(Ethyloxy)carbonyl]-1H-Pyrrolo[2,3-b]pyridin-4-yl}amino)phenyl]amino}carbonyl)cyclopropanecarboxylic acid

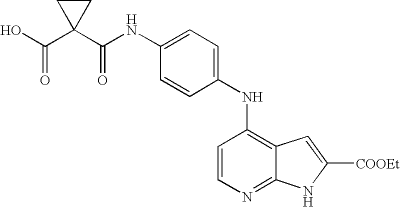

To a stirring solution of 1,1-cyclopropanedicarboxylic acid (commercially available, for example, from Sigma-Aldrich) (65.1 mg 0.5 mmol) in tetrahydrofuran (5 mL) under argon was added triethyl amine (50.6 mg, 0.5 mmol). After stirring 30 min at 0° C., thionyldichloride (59.5 mg, 0.5 mmol) was added. The mixture was warmed to room temperature, and stirred for another 10 min. Then ethyl 4-[(4-aminophenyl)amino]-1H-pyrrolo[2,3-b]pyridine-2-carboxylate (Step C) (133.3 mg, 0.45 mmol) was added to the above solution, and the mixture was stirred at room temperature for 1 h. After evaporating the solvent in vacuo, the residue was washed with methanol to give the title compound. ¹H NMR (400 MHz, DMSO-d⁶) ppm 1.33 (t, J=7.2 Hz, 3H), 1.41 (s, 4H), 4.31 (q, J=7.2 Hz, 2H), 6.59 (d, J=5.6 Hz, 1H), 7.27 (d, J=8.8 Hz, 2H), 7.48 (s, 1H), 7.62 (d, J=8.8 Hz, 2H), 8.00 (d, J=5.6 Hz, 1H), 8.91 (s, 1H), 10.81 (s, 1H), 12.13 (brs, 1H).

Step E: Ethyl 4-[(4-{[(1-{[(4-fluorophenyl)amino]carbonyl}cyclopropyl)carbonyl]amino}phenyl)amino]-1H-pyrrolo[2,3-b]pyridine-2-carboxylate (title compound)

To a solution of 1-({[4-({2-[(ethyloxy)carbonyl]-1H-pyrrolo[2,3-b]pyridin-4-yl}amino)phenyl]amino}carbonyl)cyclopropanecarboxylic acid (Step D) (163.3 mg, 0.4 mmol) in N,N-dimethylformamide (10 mL) were added diisopropylethyamine (62 mg, 0.5 mmol), o-benzotriazole-1-yl-N,N,N¹,N¹-tetramethyluronium hexafluorophosphate (182.0 mg, 0.5 mmol), and 4-fluoroaniline (commercially available, for example, from Sigma-Aldrich) (53.3 mg, 0.5 mmol). The mixture was stirred at room temperature for 1 h, and directly charged into SCX cartridge. After SCX purification, the residue was washed with methanol to afford the title compound ¹H NMR (400 MHz, DMSO-d⁶) ppm 1.33 (t, J=7.1 Hz, 3H), 1.46 (s, 4H), 4.31 (q, J=7.1 Hz, 2H), 6.59 (d, J=5.6 Hz, 1H), 7.15 (dd, J=8.8, 8.8 Hz, 2H), 7.26 (d, J=8.8 Hz, 2H), 7.48 (d, J=2.0 Hz, 1H), 7.60-7.67 (m, 4H), 8.01 (d, J=5.6 Hz, 1H), 8.89 (s, 1H), 10.05 (s, 1H), 10.08 (s, 1H), 12.11 (brs, 1H).

### Example 18

### N¹-[4-({2-[(Cyclopropylamino)carbonyl]-1H-pyrrolo[2,3-b]pyridin-4-yl}amino)phenyl]-N¹-(4-fluorophenyl)-1,1-cyclopropanedicarboxamide

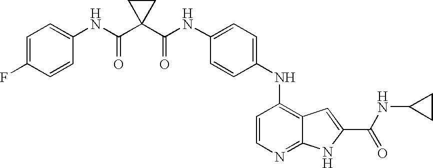

Step A: 4-[(4-{[(1-{[(4-Fluorophenyl)amino]carbonyl}cyclopropyl)carbonyl]amino}phenyl)amino]-1H-pyrrolo[2,3-b]pyridine-2-carboxylic acid

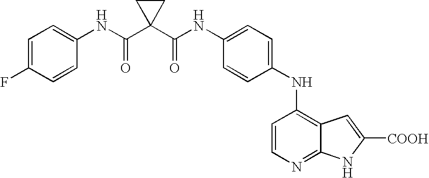

To a solution of ethyl 4-[(4-{[(1-{[(4-fluorophenyl)amino]carbonyl}cyclopropyl)carbonyl]amino}phenyl)amino]-1H-pyrrolo[2,3-b]pyridine-2-carboxylate (Example 17, 150 mg, 0.02 mmol) in methanol (10 mL), was added 6 N aqueous sodium hydroxide (0.2 mL). The mixture was stirred at 60° C. for 1 h. After adding 6 N aqueous hydrogen chloride (0.2 mL), the mixture was purified by SCX cartridge, and concentrated in vacuo to give the title compound, which was used for the next step.

Step B: N¹-[4-({2-[(Cyclopropylamino)carbonyl]-1H-pyrrolo[2,3-b]pyridin-4-yl}amino) phenyl]-N¹-(4-fluorophenyl)-1,1-cyclopropanedicarboxamide (title compound)

To a solution of 4-[(4-{[(1-{[(4-fluorophenyl)amino]carbonyl}cyclopropyl) carbonyl]amino}phenyl)amino]-1H-pyrrolo[2,3-b]pyridine-2-carboxylic acid (Step A) (30 mg, 0.06 mmol) in N,N-dimethylformamide (2 mL) were added o-benzotriazole-1-yl-N,N,N¹,N¹-tetramethyluronium hexafluorophosphate (28.8 mg, 0.08 mmol), diisopropylethyamine (9.8 mg, 0.07 mmol), and cyclopropylamine (commercially available, for example from Sigma-Aldrich) (43.4 mg, 0.08 mmol). The mixture was stirred at room temperature for 24 h, and directly purified by LC/MS to give the title compound. ¹H NMR (400 MHz, DMSO-d⁶) ppm 0.53-0.58 (m, 2H), 0.69-0.75 (m, 2H), 1.47 (s, 4H), 2.81-2.89 (m, 1H), 6.59 (d, J=5.6 Hz, 1H), 7.11-7.26 (m, 5H), 7.60 (d, J=8.8 Hz, 2H), 7.64 (dd, J=5.1, 9.1 Hz, 2H), 7.95 (d, J=5.6 Hz, 1H), 8.29 (d, J=4.0 Hz, 1H), 8.50 (s, 1H), 8.77 (s, 1H), 10.03 (s, 1H), 10.10 (s, 1H), 11.61 (br s, 1H).

### Example 19

### N¹-(4-Fluorophenyl)-N¹-(4-{[2-({[2-(4-morpholinyl)ethyl]amino}carbonyl)-1H-pyrrolo[2,3-b]pyridin-4-yl]amino}phenyl)-1,1-cyclopropane dicarboxamide

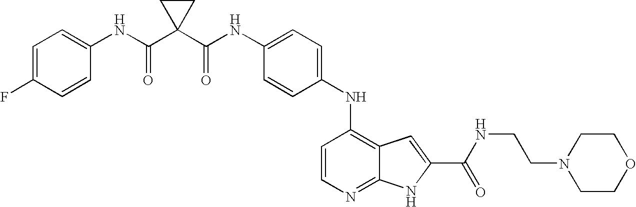

A similar procedure as Example 18 was used, with [2-(4-morpholinyl)ethyl]amine

(commercially available, for example, from Sigma-Aldrich) being substituted for cyclopropylamine to prepare the title compound. ¹H NMR (400 MHz, DMSO-d⁶) ppm 1.47 (s, 4H), 2.40-2.45 (m, 4H), 2.85-2.98 (m, 2H), 3.38-3.44 (m, 2H), 3.56-3.61 (m, 4H), 6.59 (d, J=5.6 Hz, 1H), 7.15 (dd, J=8.8, 8.8 Hz, 1H), 7.19 (s, 1H), 7.24 (d, J=8.8 Hz, 2H), 7.57-7.67 (m, 4H), 7.96 (d, J=5.6 Hz, 1H), 8.14-8.17 (m, 1H), 8.79 (s, 1H), 10.03 (s, 1H), 10.09 (s, 1H), 11.72 (brs, 1H).

### Example 20

### N¹-(4-Fluorophenyl)-N¹-(4-{[2-({[2-(1H-imidazol-4-yl)ethyl]amino}carbonyl)-1H-pyrrolo[2,3-b]pyridin-4-yl]amino}phenyl)-1,1-cyclopropane dicarboxamide

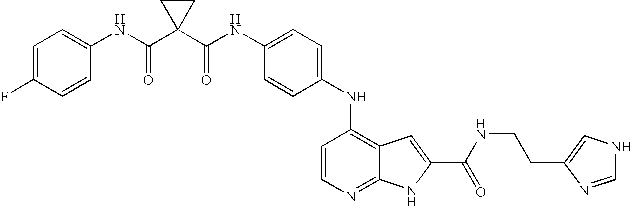

A similar procedure as Example 18 was used, with [2-(1H-imidazol-4-yl)ethyl]amine (commercially available, for example, from Sigma-Aldrich) being substituted for cyclopropylamine, to prepare the title compound. ¹H NMR (400 MHz, DMSO-d⁶) ppm 1.47 (s, 4H), 2.72-2.84 (m, 2H), 3.48-3.54 (m, 2H), 6.59 (d, J=5.3 Hz, 1H), 7.15 (dd, J=8.8, 8.8 Hz, 2H), 7.20 (s, 1H), 7.24 (d, J=8.8 Hz, 2H), 7.52-7.55 (m, 1H), 7.59-7.66 (m, 4H), 7.95 (d, J=5.3 Hz, 1H), 8.33-8.36 (m, 1H), 8.79 (s, 1H), 9.96-10.19 (m, 2H), 11.70 (rs, 1H), 11.81 (br/s, 1H).

### Example 21

### N¹-(4-Fluorophenyl)-N¹-(4-{[2-({[2-(methylsulfonyl)ethyl]amino}carbonyl)-1H-pyrrolo[2,3-b]pyridin-4-yl]amino}phenyl)-1,1-cyclopropane dicarboxamide

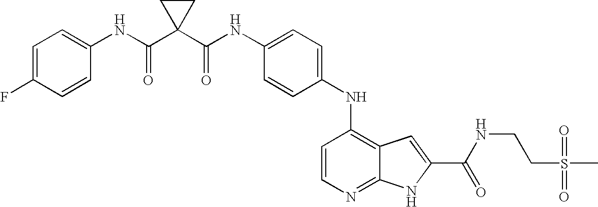

A similar procedure as Example 18 was used, with [2-(methylsulfonyl)ethyl]amine hydrochloride (commercially available, for example, from Beta Pharma) being substituted for cyclopropylamine, to prepare the title compound. ¹H NMR (400 MHz, DMSO-d⁶) ppm 1.47 (s, 4H), 3.05 (s, 3H), 3.37-3.45 (m, 2H), 3.70 (q, J=6.6 Hz, 2H), 6.58 (d, J=5.6 Hz, 1H), 7.15 (dd, J=8.8, 8.8 Hz, 2H), 7.21 (s, 1H), 7.23 (d, J=8.8 Hz, 2H), 7.58-7.67 (m, 4H), 7.97 (d, J=5.6 Hz, 1H), 8.50 (dd, J=5.6, 5.6 Hz, 1H), 8.82 (s, 1H), 10.03 (s, 1H), 10.08 (s, 1H), 11.73 (br s, 1H).

### Example 22

### N¹-(4-Fluorophenyl)-N¹-{4-[(2-phenyl-1H-pyrrolo[2,3-b]pyridin-4-yl)amino]phenyl}-1,1-cyclopropanedicarboxamide

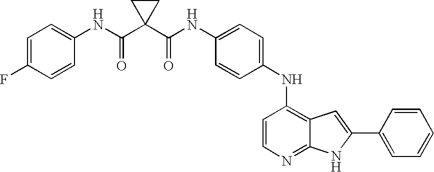

Step A: 4-Bromo-1-[(4-methylphenyl)sulfonyl]-2-phenyl-1H-pyrrolo[2,3-b]pyridine

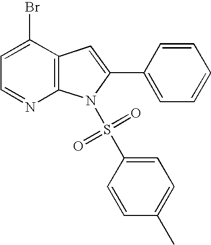

Phenylboronic acid (commercially available, for example, from Sigma-Aldrich) (243.9 mg, 2 mmol) and 4-bromo-2-iodo-1-[(4-methylphenyl)sulfonyl]-1H-pyrrolo[2,3-b]pyridine (715.7 mg, 1.5 mmol), (prepared according to methods disclosed in WO 2003 000690, were dissolved in dimethoxyethane (18 mL) and aqueous sodium carbonate (2 M, 1 mL). The resulting solution and Pd(PPh₃)₄ (173.3 mg, 0.2 mmol) were added to a microwave vial. After capping, the mixture was heated with Creator™ at 130° C. for 10 h. After that, the reaction mixture was diluted with saturated aqueous ammonium chloride, and extracted with dichloromethane (20 mL, 3 times). The organic layer was washed with brine, dried over sodium sulfate, and then evaporated to dryness under reduced pressure. The residue was purified by Yamazen Fast Flow Liquid Chromatography on a silica gel column (ethylacetate:Hexane=1 4 to 1 1) to give the corresponding product (207.2 mg, 32%). ¹H NMR (400 MHz, DMSO-d⁶) ppm 2.33 (s, 3H), 6.75 (s, 3H), 7.37 (d, J=7.8 Hz, 2H), 7.49-7.53 (m, 3H), 7.59-7.64 (m, 3H), 7.71 (d, J=8.3 Hz, 2H), 8.26 (d, J=5.3 Hz, 1H).

Step B: 4-Bromo-2-phenyl-1H-pyrrolo[2,3-b]pyridine

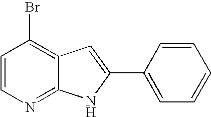

6N aqueous sodium hydroxide (0.6 mL) was added to a solution of 4-bromo-1-[(4-methylphenyl)sulfonyl]-2-phenyl-1H-pyrrolo[2,3-b]pyridine (Step A) (182.8 mg, 0.4 mmol) in methanol (10 mL). The mixture was stirred at 70° C. for 2 h. The reaction mixture was neutralized with 6N aqueous hydrogen chloride (0.6 mL), and underwent SCX purification to give the product (82.1 mg, 70%). ¹H NMR (400 MHz, DMSO-d⁶) ppm 6.92 (s, 1H), 7.34 (d, J=5.1 Hz, 1H), 7.36-7.43 (m, 1H), 7.45-7.52 (m, 2H), 7.97-8.03 (m, 2H), 8.08 (d, J=5.1 Hz, 1H), 12.54 (brs, 1H). MS (ESI): m/z 274 (M+1)+

Step C: N-(4-Nitrophenyl)-2-phenyl-1H-pyrrolo[2,3-b]pyridin-4-amine

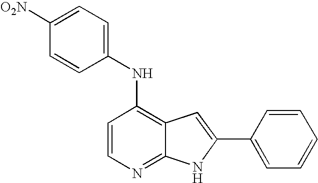

To a suspension of 4-bromo-2-phenyl-1H-pyrrolo[2,3-b]pyridine (Step B) (68 mg, 0.25 mmol), potassium tert-butoxide (56 mg, 0.5 mmol) and (commercially available for example, from Sigma-Aldrich) 4-nitroaniline (103.6 mg, 0.8 mmol) in N,N-dimethylformamide (4 mL) in a microwave vial was added Pd(dppf)Cl₂ (40.8 mg, 0.1 mmol). After capping, the mixture was heated with Creator™ at 120° C. for 30 min. After cooling down, saturated aqueous ammonium chloride was added and the resulting mixture was extracted with dichloromethane (20 mL×3 times). The organic layer was washed with brine, dried over sodium sulfate, and then evaporated to dryness under reduced pressure. The residue was used for the next step without further purification.

Step D: N-(2-Phenyl-1H-pyrrolo[2,3-b]pyridin-4-yl)-1,4-benzenediamine

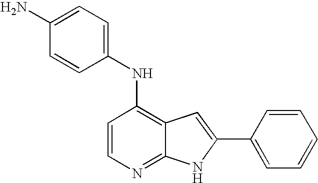

Aqueous hydrogen chloride (6 N, 1 mL) was added to a suspension of the above residue and tin (178 mg, 1.5 mmol) in ethanol (20 mL). The mixture was stirred at 80° C. for 3 h, and directly charged into SCX cartridge. After SCX purification, the residue was purified by Yamazen Fast Flow Liquid Chromatography on a silica gel column dichloromethane/methanol=1/0 TO 9/1) to give the title compound. ¹H NMR (400 MHz, DMSO-d⁶) ppm 4.99 (brs, 2H), 6.27 (d, J=5.6 Hz, 1H), 6.35 (s, 1H), 6.61 (d, J=8.6 Hz, 2H), 6.95-7.01 (m, 3H), 7.24-7.30 (m, 1H), 7.43 (dd, J=7.8, 7.8 Hz, 2H), 7.75-7.82 (m, 3H), 8.23 (s, 1H), 11.73 (brs, 1H).

Step E: 1-[({4-[(2-Phenyl-1H-pyrrolo[2,3-b]pyridin-4-yl)amino]phenyl}amino)carbonyl]cyclopropanecarboxylic acid

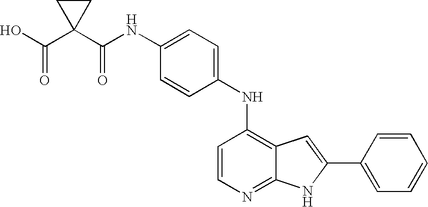

To a stirring solution of 1,1-cyclopropanedicarboxylic acid (commercially available, for example, from Sigma-Aldrich (81.9 mg 0.6 mmol) in tetrahydrofuran (6 mL) under argon was added triethylamine (63.6 mg, 0.6 mmol). After stirring 30 min at 0° C., thionyl dichloride (74.7 mg, 0.6 mmol) was added. The mixture was warmed to room temperature, and stirred for 30 min. To a solution of N-(2-phenyl-1H-pyrrolo[2,3-b]pyridin-4-yl)-1,4-benzenediamine (Step D) (50 mg, 0.2 mmol) in tetrahydrofuran (2 mL) was added the above solution portionwise, and the mixture was stirred for 3 h. The crude mixture was concentrated in vacuo, and purified by SCX cartridge. The obtained solid was washed with methanol to afford the title compound (33.1 mg, 47%). ¹H NMR (400 MHz, DMSO-d⁶) ppm 1.41 (s, 4H), 6.61 (d, J=5.6 Hz, 1H), 7.06 (d, J=1.0 Hz, 1H), 7.26 (d, J=8.8 Hz, 2H), 7.27-7.32 (m, 1H), 7.45 (dd, J=8.3, 8.3 Hz, 3H), 7.61 (d, J=8.8 Hz, 2H), 7.83 (dd, J=1.0, 8.3 Hz, 2H), 7.89 (d, J=5.6 Hz, 1H), 8.69 (s, 1H), 10.88 (s, 1H), 11.91 (br s, 1H).

Step F: N¹-(4-Fluorophenyl)-N¹-{4-[(2-phenyl-1H-pyrrolo[2,3-b]pyridin-4-yl)amino]phenyl}-1,1-cyclopropanedicarboxamide

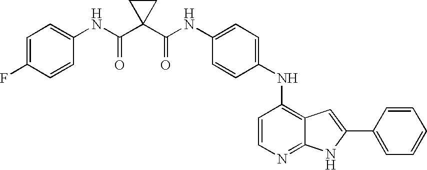

To a solution of 1-[({4-[(2-phenyl-1H-pyrrolo[2,3-b]pyridin-4-yl)amino]phenyl}amino)carbonyl]cyclopropanecarboxylic acid (Step E) (30.0 mg, 0.07 mmol) in N,N-dimethylformamide (10 mL) were added o-benzotriazole-1-yl-N,N, N′,N′-tetramethyluronium hexafluorophosphate (41.3 mg, 0.11 mmol), diisopropylethyamine (14.2 mg, 0.11 mmol), and 4-fluoroaniline (commercially available, for example, from Sigma-Aldrich) (12.2 mg, 0.11 mmol). The mixture was stirred at room temperature for 3 days, and concentrated in vacuo. The residue was purified by Yamazen Fast Flow Liquid Chromatography on a silica gel column dichloromethane/methanol=1/0 TO 9/1) to afford the title compound. ¹H NMR (400 MHz, DMSO-d⁶) ppm 1.47 (s, 4H), 6.61 (d, J=5.6 Hz, 1H), 7.05 (d, J=2.0 Hz, 1H), 7.15 (dd, J=8.8, 8.8 Hz, 2H), 7.25 (d, J=8.8 Hz, 2H), 7.26-7.33 (m, 1H), 7.45 (dd, J=7.8, 7.8 Hz, 2H), 7.59-7.67 (m, 4H), 7.83 (dd, J=1.1, 8.8 Hz, 2H), 7.89 (d, J=5.6 Hz, 1H), 8.64 (s, 1H), 10.01 (s, 1H), 10.11 (s, 1H), 11.87 (br s, 1H).

### Biological Data

### Cell-Based Assay for C-Met Autophosphorylation

### 1. Construction of the Human C-Met/cFMS Chimeric Expression Vector

To generate a chimeric cDNA clone of c-Met with cFms, the extracellular domain of colony stimulating factor 1 receptor (CSF1R encoded by cFms gene) was fused with the transmembrane and cytoplasmic domain of c-Met. Reverse-transcribed RNA from human placenta was used as template to clone c-Met. The transmembrane/cytoplasmic domain fragment of c-Met (nucleotides 933 to 1390) containing a NotI site at both 5′- and 3′-end was generated by polymerase chain reaction (PCR) using following two oligonucleotides;

5′ primer:
5′-CCCCCCGCGGCCGCCGGATTGATTGCTGGTGTTGTCTCAATATCA-
3′
3′ primer:
5′-CCCCCCGCGGCCGCCCTATGATGTCTCCCAGAAGGAGGCTGGTCG-
3′

The resulting c-Met cDNA was cloned into pCR2.1-TOPO vector (c-Met/pCR2.1-TOPO). The extracellular domain fragment of cFms (nucleotides 1 to 512) containing BamHI site at 5′-end and NotI site at 3′-end was generated from human placenta cDNA by PCR using two oligonucleotides;

5′ primer:
5′-CCCCCCGGATCCACCATGGGCCCAGGAGTTCTGCTGCTCCTGCTGG
TGGCC-3′
3′ primer:
5′-AAAAAAGGCGGCCGCCTCATCCGGGGGATGCGTGTGGGCTCCTGC-
3′

The resulting cFms cDNA was cloned into pcDNA3.1 vector (Invitrogen) using the BamHI and NotI sites (cFms/pcDNA3.1). To generate complete chimeric construct, the c-Met fragment (transmembrane and cytoplasmic domain) was digested with NotI sites (in c-Met/pCR2.1-TOPO vector) and then subcloned into cFms/pcDNA3.1 vector by NotI site (c-Met/cFms/pcDNA3.1).

### 2. Establishment of C-Met/cFMS Stable Cell Lines

NIH3T3 cells were grown in DMEM supplemented with 10% fetal bovine serum. NIH3T3 cells were transfected with c-Met/cFms/pcDNA3.1 vector alone using the calcium phosphate method according to the manufacturer's instructions. Three days after transfection, cells were selected with G418 (0.4 mgml⁻¹) for 14 days and the expression of c-Met chimeric receptor in the G418-resistant colonies of NIH3T3 (c-Met/cFms/NIH3T3) cells was analyzed by immunoblot. Autophosphorylation of c-Met induced by the stimulation with M-SCF, the ligand for CSF1R, was analyzed by immunoprecipitation and immunoblot, and prominent stable transfectant was selected for c-Met autophosphorylation assay.

### 3. Immunoprecipitation and Immunoblot Analysis

c-Met/cFms/NIH3T3 cells were grown to confluence in DMEM supplemented with 10% fetal bovine serum, 0.4 mgml⁻¹ G418 and serum starved in serum-free DMEM for 1 hour at 37° C. Cells were stimulated with M-CSF at 300 ngml⁻¹ for 10 min. Cells were washed once with cold PBS and lysed with TNE lysis buffer (10 mM Tris-HCl pH7.4, 150 mM NaCl, 1 mM EDTA, 1% NP-40, 10 mM NaF, 2 mM Na₃VO₄, 10 mM Na₄P₂O₇ and Protease Inhibitor Cocktail (Complete mini EDTA-free, Roche)). Debris and undissolved proteins were removed from cell lysates by centrifugation (15,000 rpm for 20 min at 4° C.). Cell lysates for immunoprecipitation were cleared with Protein G-Sepharose for 1 h at 4° C. and immunoprecipitated using the anti-cFms antibody overnight at 4° C. Immune complexes were then incubated with Protein G-Sepharose for 1 h at 4° C. Protein G immunoprecipitates were washed five times in TNE lysis buffer. Immunoprecipitates were resolved on 4-20% SDS-PAGE gels, and the proteins were transferred to PVDF membrane. For anti-phosphotyrosine immunoblot analysis, membranes were blocked with 3% BSA/PBS and blotted with anti-phosphotyrosine (clone 4G10, biotinylated) followed by HRP-conjugated streptavidin (PIERCE). Detection of protein was done by chemiluminescence using ECL plus reagent (Amersham) through exposing on X-ray films.

### 4. C-Met Autophosphorylation Assay

c-Met/cFms/NIH3T3 cells were plated at 1×10⁵ cells/well in collagen-coated 96-well microtitre plates and grown 24 h under standard culture conditions, followed by serum starvation for 1 h. The cells were incubated with compounds for 1 h at 37° C. and followed by M-CSF (600 ngml⁻¹) stimulation for 10 min at 37° C. The media was removed and the cells were lysed with 120 ul/well of lysis buffer (20 mM Tris-HCl pH8.0, 137 mM NaCl, 2 mM EDTA, 10% glycerol, 1% Triton X-100, 1 mM Na₃VO₄ and Protease Inhibitor Cocktail (Complete mini EDTA-free, Roche)). Then 100 ul/well of lysate was transferred to the antibody-coated (50 ng/well goat anti-cFms antibody in PBS) ELISA plate and incubated overnight at 4° C. Plates were washed five times with PBST (PBS containing 0.05% Tween-20). Primary antibody (biotin-conjugated anti-phosphotyrosine monoclonal antibody, PIERCE) was diluted 1:10,000 in PBST containing 1% BSA, added (100 ul/well) and incubated for 2 h at room temperature. After washing the plates five times with washing buffer, 100 ul/well of HRP-conjugated streptavidin (PIERCE) in PBST containing 1% BSA was added and incubated for 30 min at room temperature. After washing five times with PBST, 100 ul/well of SuperSignal ELISA Femto Substrate (PIERCE) was added and incubated for almost 1 min at room temperature. Chemiluminescence was measured using Wallac 1420 multilabel counter.

### 5. Data Analysis

The IC₅₀ was determined by using XLfit software (IDBS) with four-parameters, sigmoidal dose-response equation.

### In Vitro Screen

### 1. Source of Substrate Peptide

The peptide substrate, Biotin-aminohexyl-EEEEYFELVAKKKK-amide was purchased from SynPep Solid sample is dissolved at approximately 2.5 mM in water (concentration determined by amino acid analysis) and aliquots stored at −20° C.

### 2. Source of Enzyme

Met Kinase: A fusion protein consisting of His6-tagged Glutathione-S-Transferase (GST) and amino acid residues 956-1390 of human Met Kinase (aa 956-1390 of Entrez Protein Accession # EAL24359.1 (met proto-oncogene (hepatocyte growth factor receptor) [Homo sapiens])) from “www.ncbi.nlm.nih.gov/entrez/”) is purified from baculovirus expression system in Sf9 cells using Ni chelate column, GSH column, followed by size exclusion chromatography. Purity greater than 90%, estimated by SDS-PAGE, is achieved. Samples in 25 mM HEPES pH 7.5, 100 mM NaCl, 0.1 mM EDTA are stored at −80° C. until use.

### 3. Kinase Assay of Purified Met Kinase

Assays are performed in 96 well (Costar, Catalog No. 3789) or 384 well plates (Costar, Catalog No. 3705). Assay conditions for the peptide phosphorylation reaction (in 10, 20, 25, or 40 μl volume) mix are 100 mM Hepes buffer, pH 7.4; 0.1 mgml⁻¹ BSA; 5 mM MgCl₂; 1 mM DTT; 10 μM ATP; purified Met (1 nM final); and 1 μM peptide substrate. Compounds, titrated in DMSO, are evaluated at concentrations ranging from 50 μM to 0.2 nM. Concentrations of DMSO do not exceed 5%, resulting in less than 15% loss of Met activity relative to controls without DMSO. Reactions are incubated for 1 hour at room temperature and are stopped by addition of detection reagents containing, at final detection volume, 12.5 mM EDTA; 100 mM Hepes; 0.1 mg/ml BSA; 8 nM Streptavidin APC (Perkin Elmer catalog #CR130-150); 1 nM Europium-labelled anti-phosphotyrosine antibody (Perkin Elmer catalog #AD0067). Under the assay conditions defined above, the Km (apparent) for ATP is determined to be 40 μM.

### 4. Data Analysis

The data for compound dose responses were plotted as % Inhibition, calculated with the data reduction formula 100*(1−[(U1−C2)/(C1−C2)]), versus concentration of compound, where U is the unknown value, C1 is the average control value obtained for DMSO, and C2 is the average control value obtained for 0.05M EDTA. Data were fitted to the curve described by: y=((Vₘₐₓ*x)/(K+x)) where Vₘₐₓ is the upper asymptote and K is the IC₅₀. The results for each compound were recorded as pIC₅₀ calculated as follows: pIC₅₀=−Log 10(K).

### Results

### Cell-Based Results

The compounds of the present invention were found to have IC₅₀ values at the c-Met receptor of greater than approximately 6.0. Examples 13, 16, 20, 21 and 22 had IC₅₀ values between approximately 4.5 and 5.5. Examples 3, 11, 12, 14, 17 and 19 had IC₅₀ values between approximately 5.5 and 6.0.

### In Vitro Screen Results

The compounds of the present invention were found to have pIC₅₀ values at the c-Met receptor of greater than approximately 7, particularly the compounds of examples 1, 4, 5, 7, 8, 9 and 14 had pIC₅₀ values of greater than 8. The compounds of examples 15 and 16 had pIC₅₀ values of between approximately 6 and 7.

What is claimed is:

1. A compound of Formula (I): 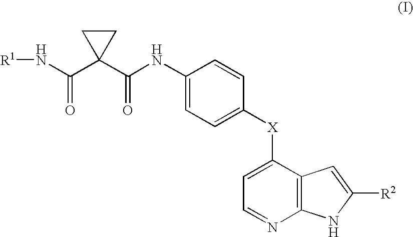
wherein
R¹ represents aryl, in which said aryl may be optionally substituted with one two or three substituents independently selected from C₁₋₆alkyl, C₁₋₆alkoxy, halogen, hydroxy or C₁₋₃haloalkyl;
R² represents hydrogen, aryl, —COOR³ or C(O)NR⁴R⁵, in which said aryl may be optionally substituted with one two or three substituents independently selected from C₁₋₆alkyl, C₁₋₆alkoxy, halogen, hydroxy or C₁₋₃haloalkyl;
X represents NH or O;
R³ represents hydrogen or C₁₋₆alkyl;
R⁴ represents hydrogen or C₁₋₆alkyl;
R⁵ represents-C₁₋₃alkylNR⁶R⁷, —C₁₋₃alkyl-SO₂—C₁₋₃alkyl, —C₁₋₃alkylOH, —C₁₋₃alkyl-C(O)NH₂, —C₁₋₃alkylheteroaryl, —C₀₋₃alkylaryl (in which said aryl may be optionally substituted with one two or three substituents independently selected from C₁₋₆alkyl, C₁₋₆alkoxy, halogen, hydroxy, C₁₋₃haloalkyl or —C₀₋₃alkylNR⁸R⁹), or C₃₋₆cycloalkyl;
R⁶ and R⁷ are each independently selected from hydrogen, C₁₋₃alkyl or together may form a 4-7 membered saturated heterocyclic ring, optionally in which one or two carbon atoms may be replaced with either oxygen or nitrogen;
R⁸ and R⁹ are each independently selected from hydrogen or C₁₋₆alkyl;
or a salt thereof.

2. A compound according to claim 1 in which R¹ represents phenyl, in which said phenyl may be optionally substituted with one or two substituents independently selected from C₁₋₃alkyl, C₁₋₃alkoxy, halogen, hydroxy or C₁₋₃haloalkyl.

3. A compound according to claim 1 in which R¹ represents phenyl, in which said phenyl may be optionally substituted with one substituent selected from methyl, methoxy, chlorine, fluorine, hydroxy or trifluoromethyl and R² is either —COOR³ or C(O)NR⁴R⁵.

4. A compound according to claim 1 in which R¹ represents phenyl substituted in the 4-position (para) with fluorine.

5. A compound according to claim 1 in which R² represents unsubstituted aryl, —COOR³ or C(O)NR⁴R⁵.

6. A compound according to claim 1 in which R⁵ represents —C₁₋₃alkylNR⁶R⁷, —C₁₋₃alkyl-SO₂—C₁₋₃alkyl, C₁₋₃alkylOH, —C₁₋₃alkyl-C(O)NH₂, —C₁₋₃alkylheteroaryl, —C₀₋₃alkylphenyl (in which said phenyl may be optionally substituted with one or two substituents independently selected from C₁₋₃alkyl, C₁₋₃alkoxy, halogen, hydroxy, C₁₋₃haloalkyl or —C₀₋₃alkylNR⁸R⁹), cyclopropane or cyclobutane.

7. A compound according to claim 6 in which the phenyl groups of R⁵ may be substituted with one or two substituents independently selected from methyl, methoxy, fluorine, chlorine, hydroxy, —CF₃ or —C₀₋₃alkylNR⁸R⁹.

8. A compound according to claim 6 in which R⁵ is selected from 2-(dimethylamino)ethyl, 2-(methylsulfonyl)ethyl, 2-(4-morpolinyl)ethyl, 2-hydroxyethyl, 2-amino-2-oxoethyl, 2-(1H-imadozl-4-yl)ethyl, 2-[(3-hydroxy-4-(methyloxy)phenyl]ethyl, [4-(dimethylamino)phenyl]methyl, [2-(methyloxy)phenyl]methyl, 2-(methyloxy)phenyl, 3-[(dimethylamino)methyl]phenyl, 3-(dimethylamino)propyl, 2-(1-piperazinyl)ethyl or cyclopropyl groups.

9. A compound according to claim 1 in which R⁶ and R⁷ are each independently selected from hydrogen, C₁₋₃alkyl or together may form a 5-7 membered ring, optionally in which one or two carbon atoms may be replaced with either oxygen or nitrogen.

10. A compound according to claim 1 in which R⁶ and R⁷ are each independently selected from hydrogen, methyl or together may form a 6-membered ring, optionally in which one carbon atom may be replaced with either oxygen or nitrogen.

11. A compound according to claim 1 in which R⁸ and R⁹ are each independently selected from hydrogen, methyl or ethyl.

12. A compound according to claim 1 in which R⁸ and R⁹ are each independently selected from hydrogen or methyl.

13. A compound which selected from:
Ethyl 4-[(4-{[(1-{[(4-fluorophenyl)amino]carbonyl}cyclopropyl)carbonyl]amino}phenyl) oxy]-1H-pyrrolo[2,3-b]pyridine-2-carboxylate;
4-[(4-{[(1-{[(4-fluorophenyl)amino]carbonyl}cyclopropyl)carbonyl]amino}phenyl)oxy]-1H-pyrrolo[2,3-b]pyridine-2-carboxylic acid;
N¹-(4-fluorophenyl)-N¹-(4-{[2-({[2-(methylsulfonyl)ethyl]amino}carbonyl)-1H-pyrrolo[2,3-b]pyridin-4-yl]oxy}phenyl)-1,1-cyclopropane dicarboxamide;
N¹-(4-{[2-({[2-(dimethylamino)ethyl]amino}carbonyl)-1H-pyrrolo[2,3-b]pyridin-4-yl]oxy}phenyl)-N¹-(4-fluorophenyl)-1,1-cyclopropanedicarboxamide;
N¹-(4-fluorophenyl)-N¹-(4-{[2-({[2-(1-piperazinyl)ethyl]amino}carbonyl)-1H-pyrrolo[2,3-b]pyridin-4-yl]oxy}phenyl)-1,1-cyclopropane dicarboxamide;
N¹-(4-{[2-({[3-(dimethylamino)propyl]amino}carbonyl)-1H-pyrrolo[2,3-b]pyridin-4-yl]oxy}phenyl)-N¹-(4-fluorophenyl)-1,1-cyclopropane dicarboxamide;
N¹-{4-[(2-{[[2-(dimethylamino)ethyl](methyl)amino]carbonyl}-1H-pyrrolo[2,3-b]pyridin-4-yl)oxy]phenyl}-N¹-(4-fluorophenyl)-1,1-cyclopropane dicarboxamide;
N¹-(4-fluorophenyl)-N¹-{4-[(2-{[(2-hydroxyethyl)amino]carbonyl}-1H-pyrrolo[2,3-b]pyridin-4-yl)oxy]phenyl}-1,1-cyclopropanedicarboxamide;
N¹-{4-[(2-{[(2-amino-2-oxoethyl)amino]carbonyl}-1H-pyrrolo[2,3-b]pyridin-4-yl)oxy]phenyl}-N¹-(4-fluorophenyl)-1,1-cyclopropanedicarboxamide;
N¹-(4-fluorophenyl)-N¹-(4-{[2-({[2-(1H-imidazol-4-yl)ethyl]amino}carbonyl)-1H-pyrrolo[2,3-b]pyridin-4-yl]oxy}phenyl)-1,1-cyclopropane dicarboxamide;
N¹-(4-fluorophenyl)-N¹-[4-({2-[({2-[3-hydroxy-4-(methyloxy)phenyl]ethyl}amino) carbonyl]-1H-pyrrolo[2,3-b]pyridin-4-yl}oxy)phenyl]-1,1-cyclopropanedicarboxamide;
N¹-[4-({2-[({[4-(dimethylamino)phenyl]methyl}amino)carbonyl]-1H-pyrrolo[2,3-b]pyridin-4-yl}oxy)phenyl]-N′-(4-fluorophenyl)-1,1-cyclopropane dicarboxamide;
N¹-(4-fluorophenyl)-N¹-[4-({2-[({[2-(methyloxy)phenyl]methyl}amino)carbonyl]-1H-pyrrolo[2,3-b]pyridin-4-yl}oxy)phenyl]-1,1-cyclopropane dicarboxamide;
N¹-(4-fluorophenyl)-N¹-(4-{[2-({[2-(methyloxy)phenyl]amino}carbonyl)-1H-pyrrolo[2,3-b]pyridin-4-yl]oxy}phenyl)-1,1-cyclopropane dicarboxamide;
N¹-[4-({2-[({3-[(dimethylamino)methyl]phenyl}amino)carbonyl]-1H-pyrrolo[2,3-b]pyridin-4-yl}oxy)phenyl]-N′-(4-fluorophenyl)-1,1-cyclopropane dicarboxamide;
N¹-(4-fluorophenyl)-N¹-[4-(1H-pyrrolo[2,3-b]pyridin-4-ylamino) phenyl]-1,1-cyclopropanedicarboxamide;
N¹-phenyl-N¹-[4-(1H-pyrrolo[2,3-b]pyridin-4-ylamino)phenyl]-1,1-cyclopropane dicarboxamide;
ethyl 4-[(4-{[(1-{[(4-fluorophenyl)amino]carbonyl}cyclopropyl) carbonyl]amino}phenyl)amino]-1H-pyrrolo[2,3-b]pyridine-2-carboxylate;
N¹-[4-({2-[(cyclopropylamino)carbonyl]-1H-pyrrolo[2,3-b]pyridin-4-yl}amino)phenyl]-N¹-(4-fluorophenyl)-1,1-cyclopropanedicarboxamide;
N¹-(4-fluorophenyl)-N¹-(4-{[2-({[2-(4-morpholinyl)ethyl]amino}carbonyl)-1H-pyrrolo[2,3-b]pyridin-4-yl]amino}phenyl)-1,1-cyclopropane dicarboxamide;
N¹-(4-fluorophenyl)-N¹-(4-{[2-({[2-(1H-imidazol-4-yl)ethyl]amino}carbonyl)-1H-pyrrolo[2,3-b]pyridin-4-yl]amino}phenyl)-1,1-cyclopropane dicarboxamide;
N¹-(4-fluorophenyl)-N¹-(4-{[2-({[2-(methylsulfonyl)ethyl]amino}carbonyl)-1H-pyrrolo[2,3-b]pyridin-4-yl]amino}phenyl)-1,1-cyclopropane dicarboxamide;
N¹-(4-fluorophenyl)-N¹-{4-[(2-phenyl-1H-pyrrolo[2,3-b]pyridin-4-yl)amino]phenyl}-1,1-cyclopropanedicarboxamide;
or a salt thereof.

14. A compound which is N¹-(4-Fluorophenyl)-N¹-(4-{[2-({[2-(4-morpholinyl)ethyl]amino}carbonyl)-1H-pyrrolo[2,3-b]pyridin-4-yl]oxy}phenyl)-1,1-cyclopropane dicarboxamide or a salt thereof

15-20. (canceled)

21. A composition which comprises a compound according to claim 1, optionally with one or more pharmaceutically acceptable carriers and/or excipients.

22. A combination comprising a compound according to claim 1, and one or more other therapeutic agents.

23. The combination according to claim 22, further comprising at least one additional anti-cancer agent.

24-35. (canceled)
